SEQUENCE LISTING (9 sequences)

SEQ ID NO: 1 - DNA, 20 bp, Artificial Sequence
  Synthetic
atcagatgacatagatatat

SEQ ID NO: 2 - DNA, 19 bp, Artificial Sequence
  Synthetic
atagatcgatgatgaggac

SEQ ID NO: 3 - protein, 82 aa, Artificial Sequence
  Synthetic
LAKLVLNSLYGKFASNPDVTGKVPYLKENGALGFRLGEEETKDPVYTPMGVFITAWARYTTITAAQACYDRIIYCDTDSIHL

SEQ ID NO: 4 - protein, 82 aa, Artificial Sequence
  Synthetic
MAKLVLNSLYGKFGTSIDVTGKEVFLKEDGSTGFRKGQKEERDPVYMPMGAFITAYARDVTIRTAQKCYDRILYCDTDSIHL

SEQ ID NO: 5 - protein, 82 aa, Artificial Sequence
  Synthetic
LAKLQLNSLYGKFASHPDVTGKVPYLKDDGSTAFKKGLPKSKDPVYTPAGAFITAWARHMTITTAQKVYDRILYCDTDSIHI

SEQ ID NO: 6 - protein, 82 aa, Artificial Sequence
  Synthetic
LAKLMFDSLYGKFASNPDVTGKVPYLKEDGSLGFRVGDEEYKDPVYTPMGVFITAWARFTTITAAQACYDRIIYCDTDSIHL

SEQ ID NO: 7 - protein, 82 aa, Artificial Sequence
  Synthetic
NAKGMLNSLYGKFGTNPDITGKVPYMGEDGIVRLTLGEEELRDPVYVPLASFVTAWGRYTTITTAQRCFDRIIYCDTDSIHL

SEQ ID NO: 8 - protein, 82 aa, Artificial Sequence
  Synthetic
QAKLMLNSLYGKFATNPDITGKVPYLDENGVLKFRKGELKERDPVYTPMGCFITAYARENILSNAQKLYPRFIYADTDSIHV

SEQ ID NO: 9 - protein, 81 aa, Artificial Sequence
  Synthetic
IAKLHLNSLYGKFASNPNVTSKIPILKDGIVKLVRGVPEQRPPVYTAAGVFITAYARNITIRAAQANFDSFAYADTDSLHL

(19) United States
(12) Patent Application Publication
(10) Pub. No.: US 2021/0174903 A1
(43) Pub. Date: Jun. 10, 2021

(54) ENHANCED PROTEIN STRUCTURE PREDICTION USING PROTEIN HOMOLOG DISCOVERY AND CONSTRAINED DISTOGRAMS

(71) Applicant: Protein Evolution Inc., Guilford, CT (US)

(72) Inventors: Jonathan M. Rothberg, Guilford, CT (US); Brian Reed, Madison, CT (US); Eric Kauderer-Abrams, Redwood City, CA (US); Zhizhuo Zhang, Branford, CT (US); Mohammad Moghadamfalahi, Guilford, CT (US)

(73) Assignee: Protein Evolution Inc., Guilford, CT (US)

(21) Appl. No.: 17/118,421

(22) Filed: Dec. 10, 2020

Related U.S. Application Data
(60) Provisional application No. 62/946,309, filed on Dec. 10, 2019.

(51) Int. Cl. G16B 30/10; G16B 15/00; G16B 5/20; C12N 15/10; G16B 40/00

(52) U.S. Cl. CPC G16B 30/10; G16B 15/00; G16B 40/00; C12N 15/1058; G16B 5/20

(57) ABSTRACT

The present disclosure provides, in some aspects, methods for enhanced protein structure prediction using protein homology discovery and constrained distograms.

### RELATED APPLICATION

This application claims the benefit under 35 U.S.C. § 119(e) of U.S. provisional application No. 62/946,309, filed Dec. 10, 2019, which is incorporated by reference herein in its entirety.

### BACKGROUND

Protein engineering is a process of developing useful or valuable proteins, or of modifying a protein by altering its chemistry, usually to improve its function for a particular application. Proteins are biological machines with many industrial and medical applications; proteins are used in detergents, cosmetics, bioremediation, industrial-scale reactions, life science research, and the pharmaceutical industry, with many modern drugs derived from engineered recombinant proteins. Solving protein structures is a fundamental step in engineering proteins.

### SUMMARY

The present disclosure provides methods for determining the three-dimensional structure of a molecule (e.g., protein). The inventors found that combining a computer-implemented protein structure prediction algorithm wherein the input protein sequences are determined using multiple sequence analysis (MSA) and at least one empirically measured distance between two amino acid residues using in vitro experiments enables accurate determination of three-dimensional protein structures at low cost and with minimal time. A first prediction of a protein structure in silico based on a protein primary structure obtained using MSA can be used to identify pairs of amino acids for analysis in an in vitro biochemical experiment. The in vitro biochemical experiment is then designed to empirically measure distances between the two amino acids in solution. These measured distances can be further utilized to constrain and refine the protein structure prediction algorithm in order to generate a second-generation prediction of the structure of the protein.

### BRIEF DESCRIPTION OF THE DRAWINGS

FIG. 1 is a flow diagram of the steps of an illustrative process for performing the methods of the present disclosure to generate a predicted protein structure. Protein homologs identified using Multiple Sequence Alignment are used as a component of input features to run a protein structure prediction algorithm. FRET-measured distances between discrete amino acid residues are used to constrain the distogram of the protein structure prediction algorithm.

FIG. 2 is a flow diagram of the steps of an illustrative process for discovering protein homologs.

FIGS. 3A-3B are flow diagrams showing steps 1 (FIG. 2A) and 2 (FIG. 2B) of an example methodology for in silico Phi29 homolog mining from the whole-genomic metagenomic fraction of the NCBI Sequence Read Archive (SRA).

FIG. 4 is a flow diagram of the steps of an illustrative process for probe design.

FIG. 5 is a schematic showing construction of a representative reference MSA for the 16S gene.

FIG. 6 includes graphs representative of an associated position-specific weight matrix (PWM) for the 16S gene example.

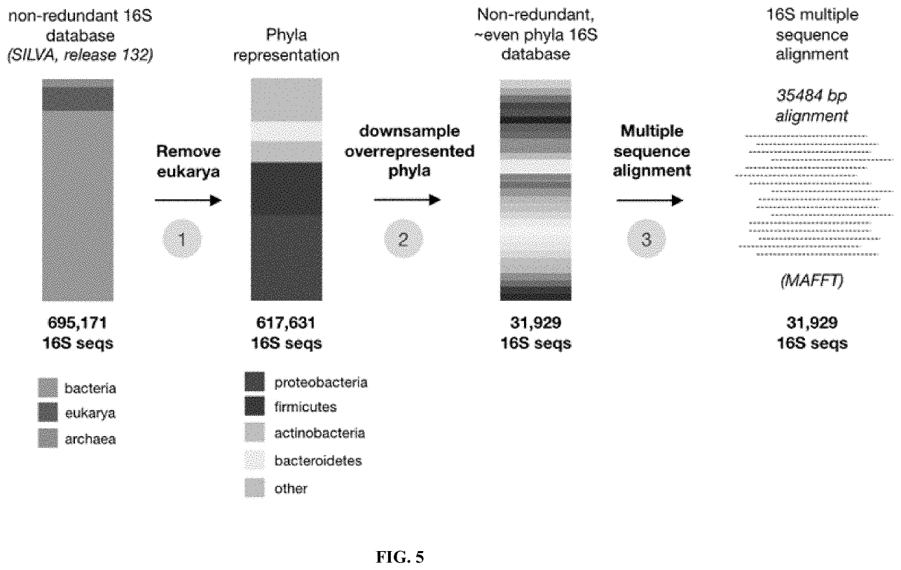

FIG. 7 is a flow diagram of the steps for candidate probe scoring and ranking for the 16S gene example.

FIG. 8 is an alignment showing a selected optimal probe set for the 16S gene. Designed optimal probes overlap with conserved regions identified by others as optimal probe regions.

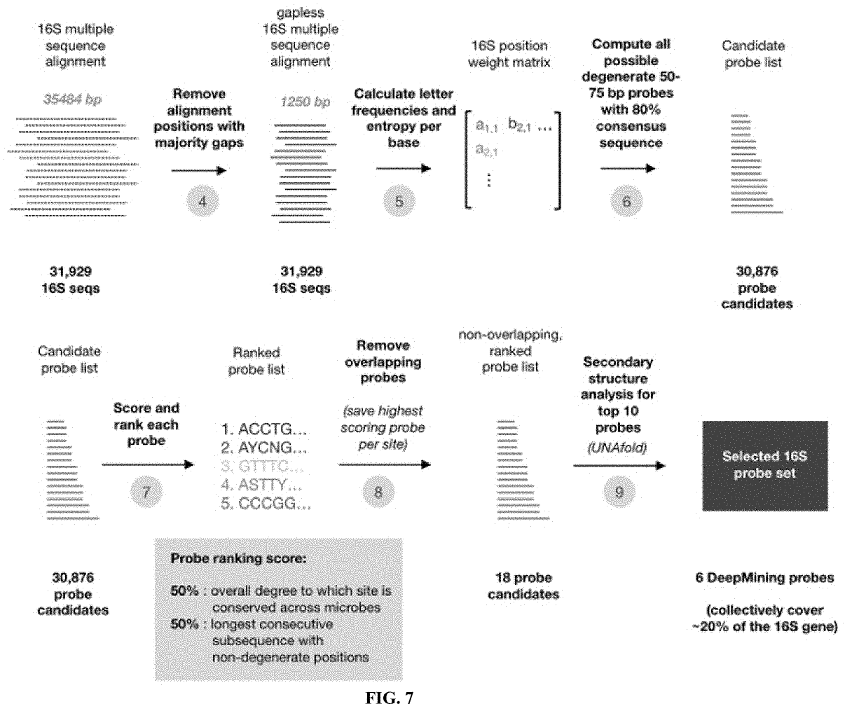

FIG. 9 is an example fragment length distribution for a tagmented soil library.

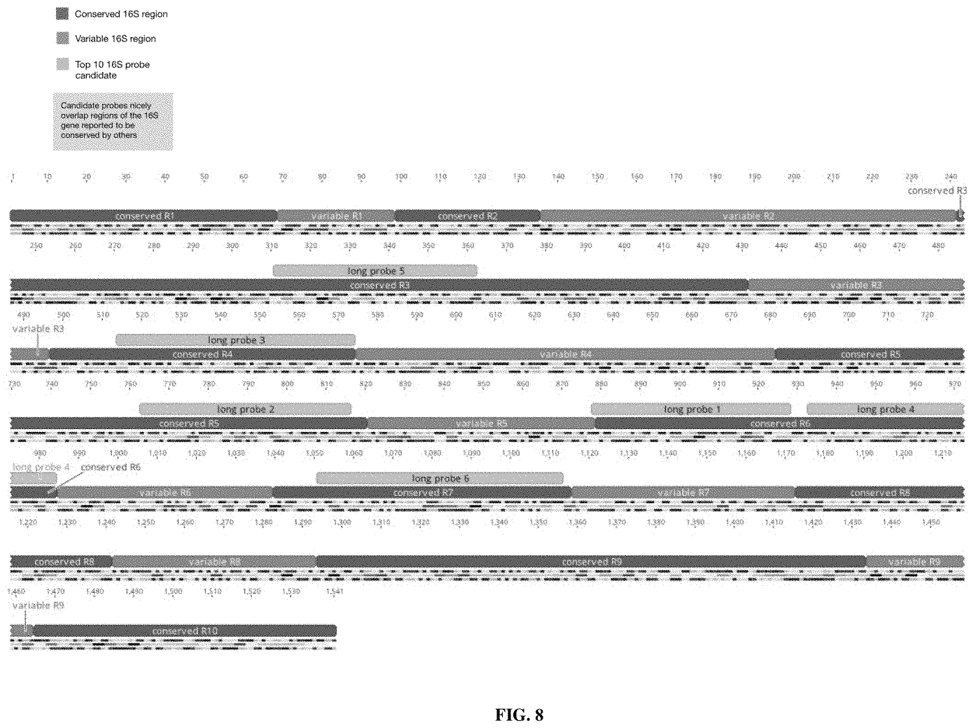

FIG. 10 includes graphs showing the results of tuning scodaphoresis parameters to control the stringency of target enrichment.

FIG. 11 is a flow diagram of the overall workflow for the example application, target enrichment by scodaphoresis.

FIG. 12 is a diagram of the scodaphoresis methodologies implemented.

FIG. 13 includes graphs showing read length statistics for pre- and post-enriched soil samples.

FIG. 14 includes graphs showing protein domain frequency in the pre and post-enriched samples.

FIG. 15A includes graphs showing quantification of enrichment across scodaphoresis methods at individual homolog level.

FIG. 15B includes graphs showing a comparison of DM and OT scodaphoresis approaches for mining divergent sequences.

FIG. 16 is a description and sample alignment of the new OT_102800 homolog.

FIG. 17 is an updated phylogeny of the Phi29 family with the newly discovered OT_102800 homolog.

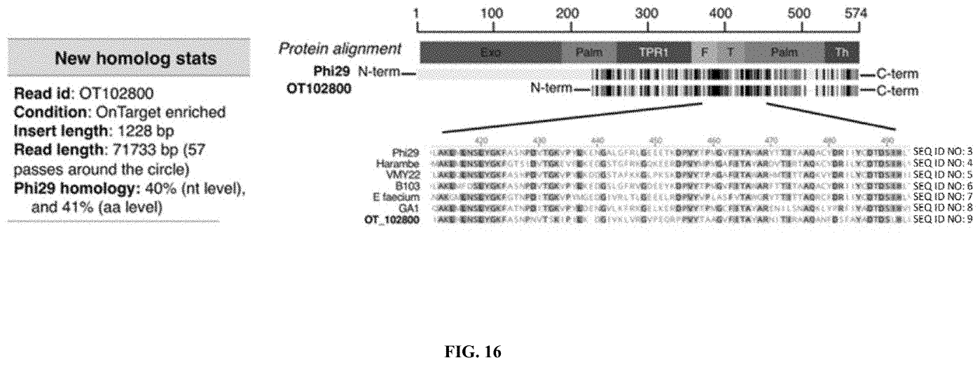

FIG. 18 is a block diagram of an illustrative implementation of a computer system for performing the methods described throughout the invention (e.g., discovery of protein homologs; determination of predicted protein structure).

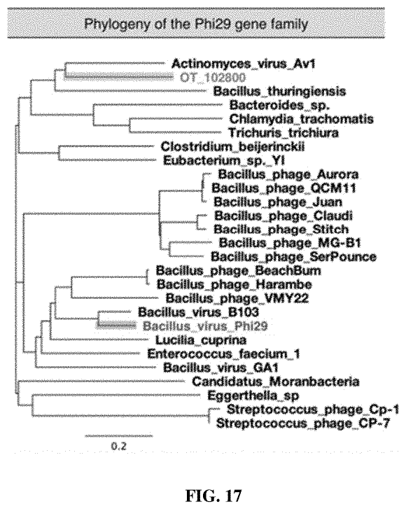

FIG. 19 is a flow diagram of the steps of an illustrative process for constraining the model using in vitro FRET measurements.

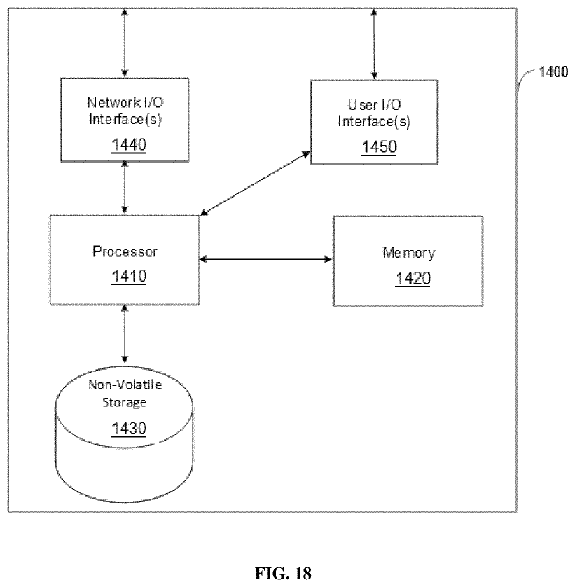

FIG. 20 is a schematic showing FRET pairs on protein structures. Multiple pairs of solvent-exposed amino acids (typically estimated to be 2-10 nanometers apart) can be selected chosen for each variant. Each pair of amino acids is labeled with FRET dye molecules on a different protein to reduce experimental cross-talk and eliminate background uncertainty.

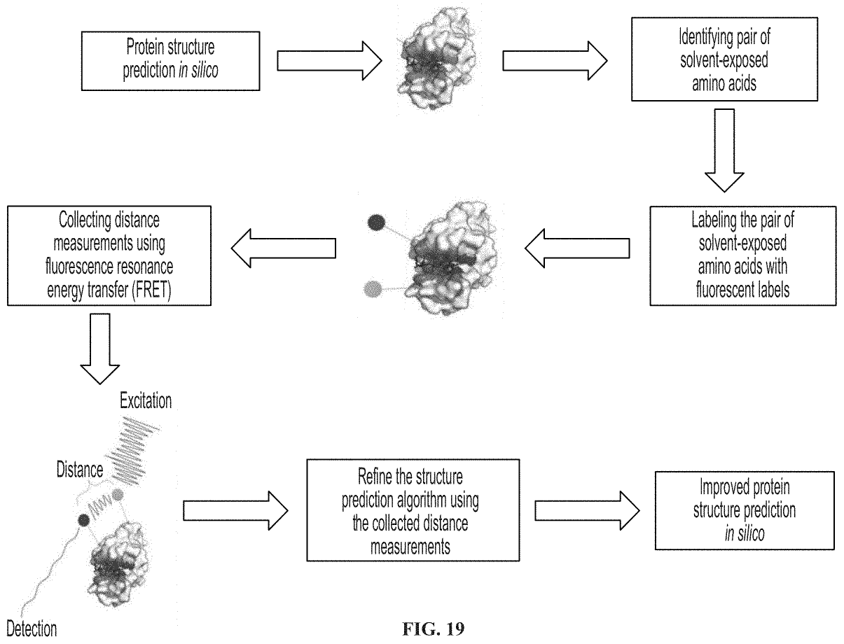

FIG. 21 is a schematic showing that, when 1:1 mixture of two FRET dye molecules (1:1 mixture of a FRET donor and a FRET acceptor) is conjugated to two exposed amino acid residues (e.g., two cysteines), there is a maximum theoretical labeling efficiency of 50% (i.e., 50% of labeled protein will have the correct pairing of FRET donor on one amino acid of the pair and FRET acceptor on the second amino acid of the pair).

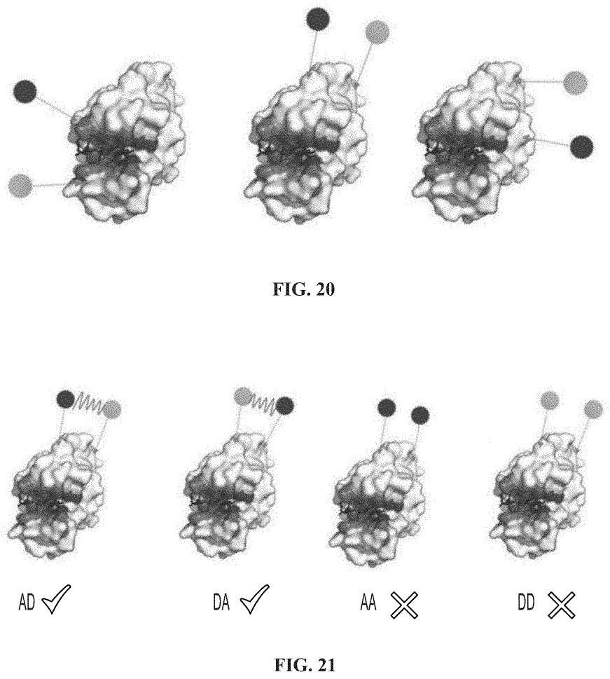

FIG. 22 is a schematic showing the process of collecting distance measurements between several pairs of amino acids using FRET and then aggregating that distance measurement data into a distogram matrix. The data in the distogram matrix can then be used to constrain and refine the protein structure prediction model.

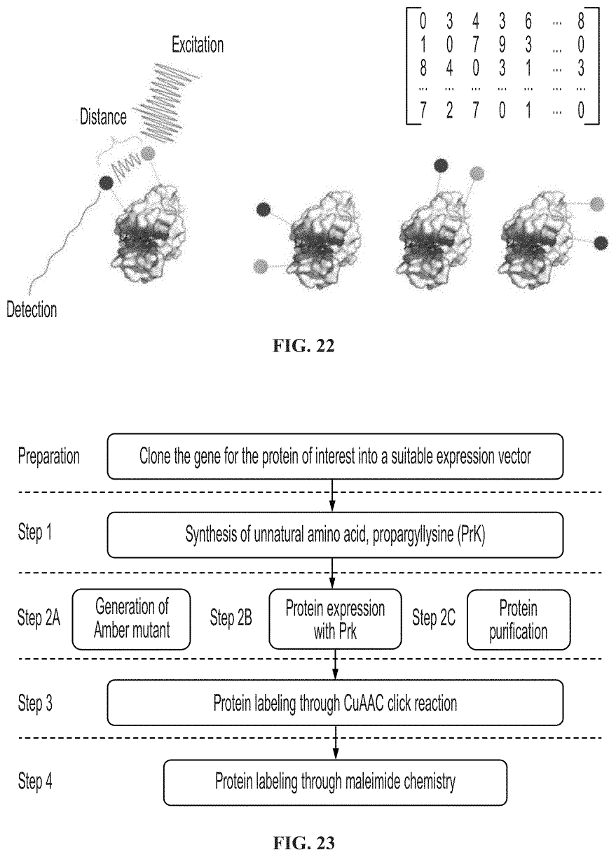

FIG. 23 is a flow diagram of an exemplary process labeling a protein with a non-natural amino acid.

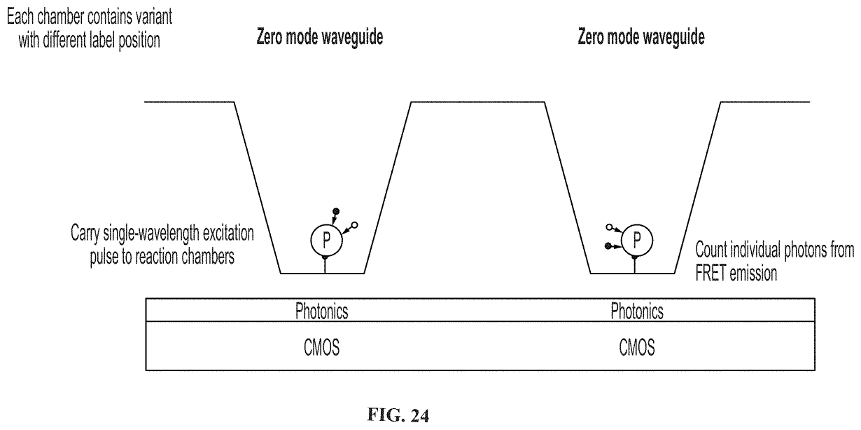

FIG. 24 is a schematic showing a zero-mode waveguide apparatus containing multiple proteins having different pairs of amino acids labeled with FRET dyes. Each protein is conjugated via a streptavidin-biotin linker to the surface of an individual chamber of the zero-mode waveguide apparatus to enable collection of distance measurements between each of the different pairs of amino acids using FRET simultaneously.

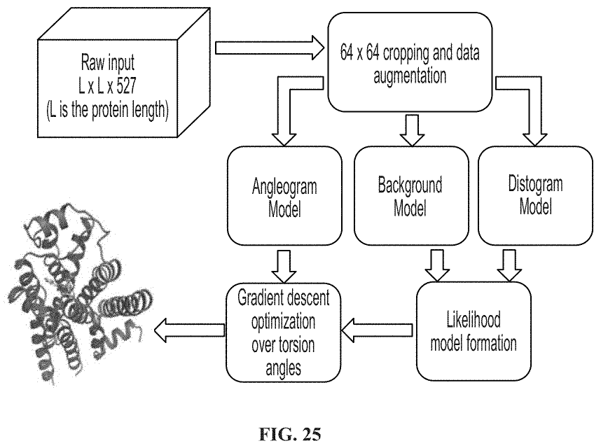

FIG. 25 is a schematic of a protein structure prediction model.

FIG. 26 is a schematic of refined components of a protein structure prediction model.

FIG. 27 is a schematic of a generative model.

FIG. 28 is a schematic showing a series of distance matrix outputs capturing the structure of the target protein, relative to random initialization.

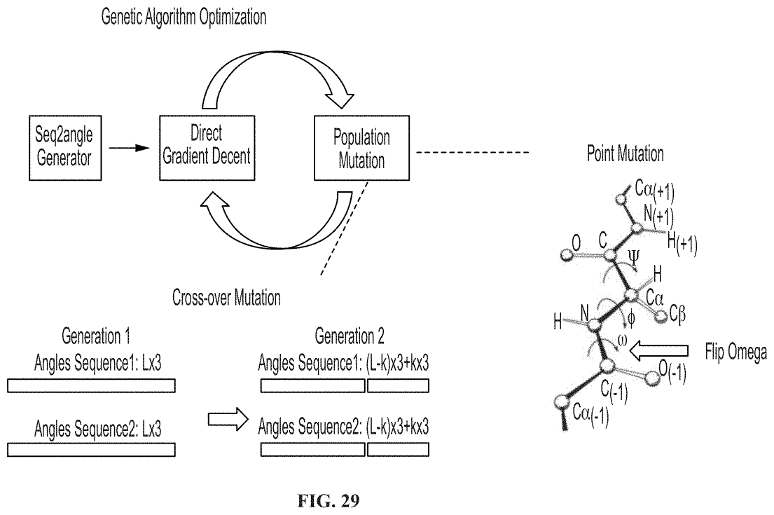

FIG. 29 is a schematic showing optimization of a genetic algorithm.

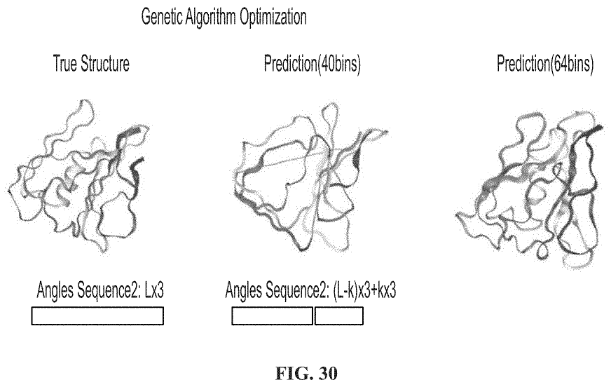

FIG. 30 is a schematic showing predicted structure outcomes following use of a genetic algorithm.

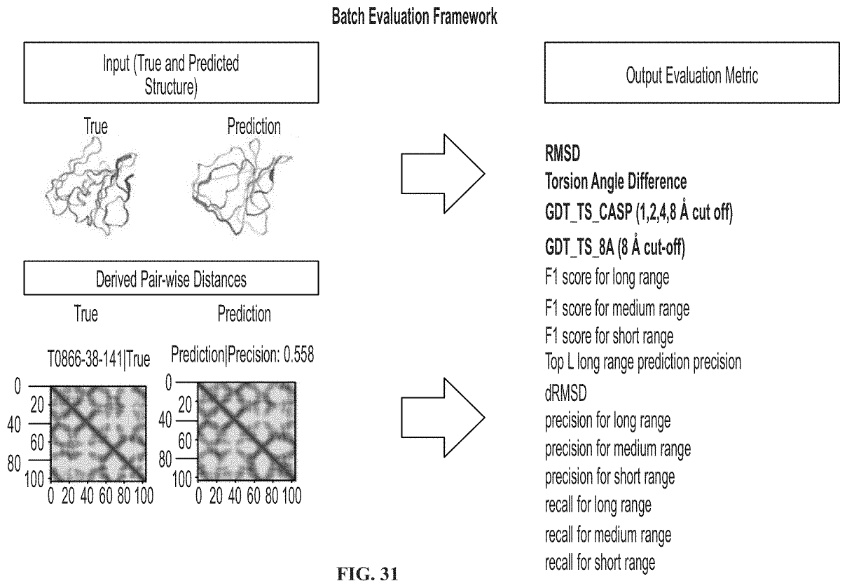

FIG. 31 is a schematic showing a framework for assessing the quality of a prediction produced by an algorithm.

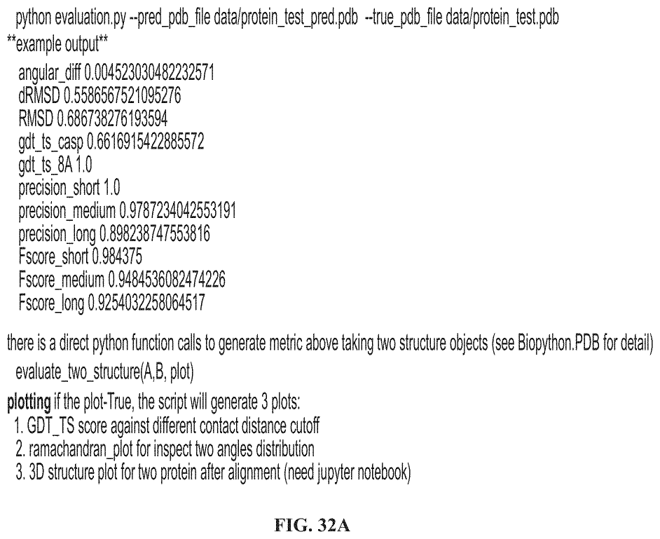

FIGS. 32A-32D are schematics showing built-in visualization allowed by a protein structure prediction algorithm.

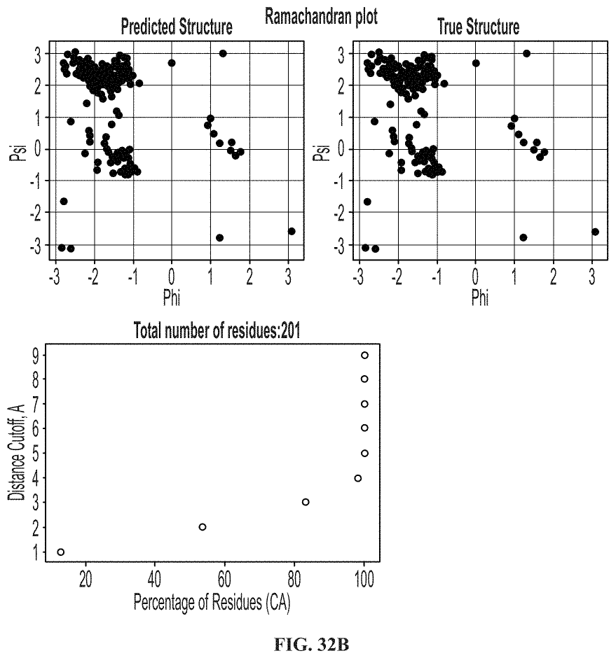

FIG. 33 is a schematic showing predicted structure from a protein structure prediction algorithm compared to the true ground-state structure.

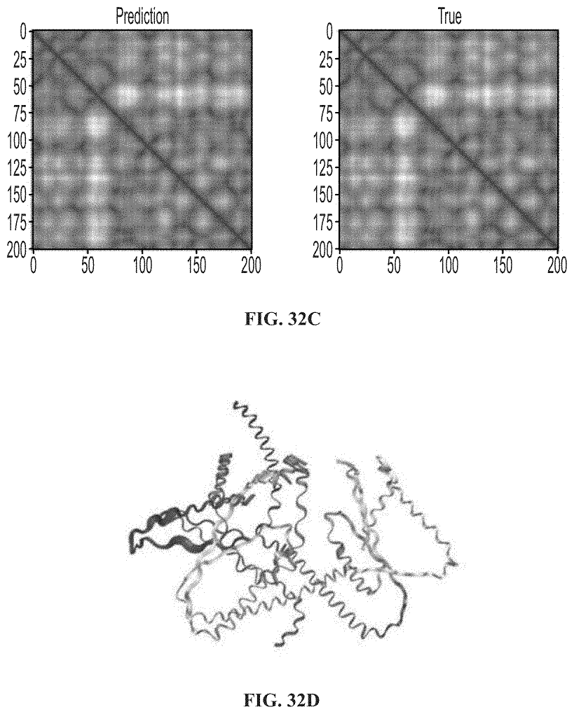

FIG. 34 is flow diagram of an illustrative process for generating new functional protein sequences.

FIG. 35 is a flow diagram illustrative of such a closed-loop, machine-learning guided platform for directed evolution.

FIG. 36 is a flow diagram illustrating an exemplary ResBlock.

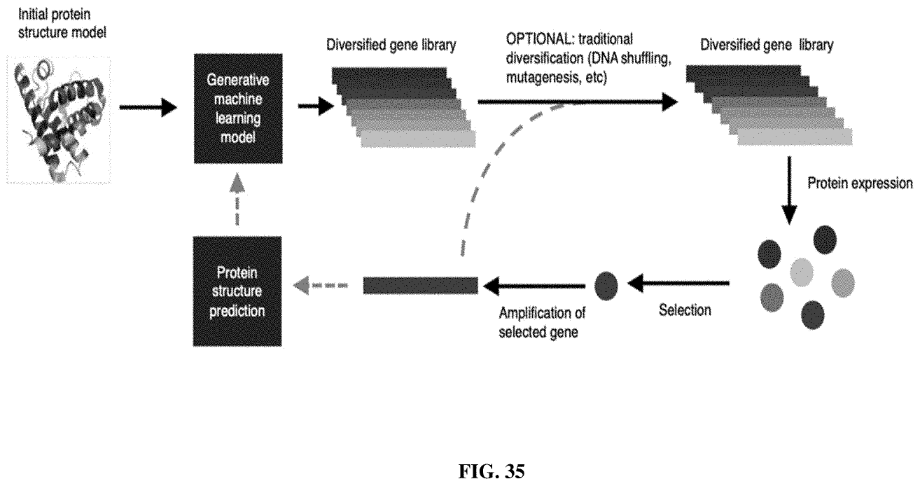

FIG. 37 is a sketch illustrating pseudo code for generating diverse (“low-identity”) functional protein sequences

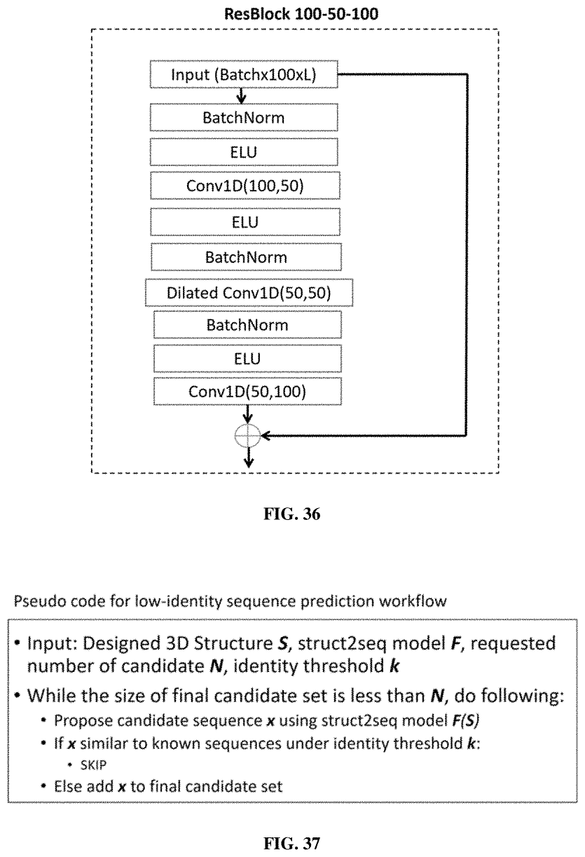

### DETAILED DESCRIPTION

The present disclosure provides systems and methods for performing molecular (e.g., protein) structure prediction using structure prediction algorithms such as AlphaFold and RaptorX. The inventors have utilized structure prediction algorithms (e.g., machine learning models) to combine protein homology discovery with improved protein structure prediction of said protein homologs.

Methods described herein generate a list of protein homologs using Multiple Sequence Alignment to produce aligned protein sequences (e.g., 1, 2, 3, 4, 5, or more aligned sequences). These sequences can be used as input sequences for a structure prediction algorithm. A feature extraction step (e.g., Direct Coupling Analysis (DCA)) will be performed to determine estimated torsion angles and distance measurements for the MSA of interest. The feature extraction stage may also include algorithms that determine information about secondary structure, exposed charge locations, and/or other biophysical details of the protein defined by the MSA. The output of the feature extraction stage will then be combined with the primary sequence for the protein and passed as input to a deep learning neural network. The deep learning network has two distinct parts—a component that computes a probability distribution over distances (called a distogram) between each pair of amino acids; and a component that computes a probability distribution over the bond and torsion angles (called an angleogram) between neighboring residues. These two components may be run independently. The final stage of the structure prediction algorithm is to sample a single structure from the probability distributions over distances and angles. This will be performed using a maximum likelihood estimate to select the configuration of angles that are most likely to occur in solution based on the probability distribution defined by the learned probability distribution over pairwise distances. From the distogram-based computational step, pairs of amino acid residues of the protein defined by the MSA will be identified. These pairs of amino acid residues will be those pairs of amino acids in the protein that could most benefit from in vitro determination of the precise distance between them (e.g., because the estimated distance produced by the algorithm is uncertain). Following in vitro measurement of these distances (e.g., using fluorescence resonance energy transfer experiments), the algorithm will be constrained such that the distances in the distogram component are fixed. This constraint will improve the stringency of the model and, upon refinement and re-running of the algorithm, is expected to produce a highly accurate predicted structure of the protein(s) as defined by the MSA.

For the majority of proteins, the primary tool for determining protein structure is X-ray crystallography, a tool that has been used to determine crystal structures of proteins since the late 1950s. To date, over 100,000 protein structures were determined at resolution better than 2 angstroms protein structures have been solved using this method. However, X-ray crystallography is time-intensive and expensive (average cost of over $50,000 per protein), is limited to protein structures that are able to form crystals, and provides a static protein structure (i.e., not a dynamic structure, as in solution).

Advances in laser-free electron lasers for hard X-rays, which produce femtosecond X-ray pulses, allows for the structural exploration of ultra-fast events in sub-picosecond time scales. However, the technique is limited to cyclic and reversible reactions triggered by light. The majority of industrial and biomedical applications of proteins involve irreversible reactions such as enzymatically catalyzed reactions. These are typically irreversible, single-pass reactions where substrates bind and are converted into product that is released from the enzyme. Limited dynamic techniques exist to study these reactions but require complex sample mixing techniques in the presence of synchrotron or XFEL x-ray sources. These methods are complex, expensive, and time-intensive to implement.

All crystallography methods are fundamentally limited to protein variants that are able to form crystals at sufficiently high concentrations. Slight variations of the same protein may have completely different crystallization conditions, and many proteins are completely unable to crystalize and are therefore unsuitable for this method.

NMR spectroscopy is also used to obtain high resolution three-dimensional structures of proteins. In contrast to X-ray crystallography, NMR spectroscopy is usually limited to very small proteins (under 35 kDa). It is used to form Conformation Activity Relationships where the structure is compared before and after interaction with a target molecule, such as a drug candidate. The technique is limited due to the crowding and overlapping of the one-dimensional spectrographic signal when larger proteins are analyzed.

Cryogenic electron microscopy (cryo-EM) is another technique for protein structure prediction. Cryo-EM does not require the crystallization of proteins, as aqueous samples of proteins are directly imaged. This greatly increases the number of protein variants that can be imaged with this technology. However, the utility of cryo-EM is currently limited to large proteins and protein complexes due to limitations in resolution. Additionally, cryo-EM is unable to capture time-resolved structures because the sample must be cryogenically frozen, preventing enzymatic activity.

No analytical technology exists to allow for benchtop protein structural determination, either static or dynamic. Such a technology would dramatically increase the speed of protein candidate screening by allowing many candidates to be screened in parallel and in rapid succession with basic laboratory equipment.

Due to the inherent challenges and competing advantages and limitations of the existing methods for empirically elucidating protein structure, there has been a longstanding interest in developing in silico approaches to determining a protein's structure from its amino acid sequence. Many in silico analyses of protein structure and function begin by identifying a protein's “homologs.” Two proteins are considered homologous if they are descended from a common ancestor. Homologous proteins can have substantially different sequences, but they often have similar function and structure. Once a protein of interest's homologs are known, there are several possible in silico routes to protein structure prediction.

In some cases, a 3D structure is not available for the protein of interest, but a 3D structure has already been experimentally gathered for an identified homolog. Since similar amino acid sequences adopt similar structures, an amino acid sequence alignment of the target protein and the homolog as well as the experimentally determined homolog's structure can be used to generate an atomic model of the target protein. This process is called “homology modeling.” If a full-length homologous protein with known structure cannot be found, one can also look for homology between small subsets of the target protein and libraries of shorter homologous sequences, each of which adopt a known fold. This “protein threading” approach can thus be used to build a structure from a collection of short homologous sequences, each contributing a little bit towards defining a portion of the overall structure.

If a protein of interest has no suitable homologous templates, ab initio methods may be used to predict the structure of the protein from amino acid sequences alone. Ab initio methods include physics-based modeling, where thermodynamic and molecular energy parameters are used to propose and rank candidate structures until a minimum entropy/maximum stability model is found.

It is also possible to infer information about a protein's three-dimensional structure by comparing the sequences of homologs and measuring the correlations in amino acid identity at pairs of residues. If two non-neighboring residues are physically in contact, for example by forming a hydrogen bond, then the amino acid identities in these positions will be correlated. Should a mutation at one position occur, it will likely be accompanied by a compensatory mutation in the other residue. In contrast, for two non-neighboring residues that are not in contact, there should be no correlation between their amino acid identities. Co-evolutionary statistical models that capture the tendency of particular pairs of residues to mutate together within a family of protein homologs can thus be used to generate “contact maps” that describe inter residue contacts protein-wide. Contact maps are an important first step towards predicting all inter-residue (pairwise) distances for the amino acids in a protein. Such a distance matrix would be completely descriptive of the 3D structure, and thus, contact maps are an important element of computational protein structure prediction.

### Combination of In Vitro FRET and in Silico Structure Prediction

Fluorescence resonance energy transfer (FRET) can be used to measure the distances between a critical amino acid residue pairs in order to improve (i.e., refine) the performance of a protein structure prediction algorithm by constraining the parameters of the algorithm. For many proteins, a difficulty in running structure prediction algorithms is caused by the existence of many plausible candidate structures that are distinct from the ground-truth structure. These plausible but incorrect candidate structures manifest as spurious local minima in the loss surface of the algorithm. The existence of many spurious local minima significantly increases the difficulty of converging to the correct structure through traditional gradient-based optimization methods. By experimentally determining the physical distances between pairs of amino acid residues of a protein in solution, the inventors of the present disclosure were able to refine a protein structure prediction algorithm in order to produce a superior prediction of individual protein structures.

First, the methods described herein utilize a structure prediction algorithm to identify pairs of amino acids for which distances should be measured (e.g., by determining the estimated distances between all pairs of amino acids using the algorithm and identifying pairs of amino acids based on at least one of several algorithm-predicted factors.

In some embodiments, an algorithm-predicted factor is the degree of variance or uncertainty in the estimated distance between a pair of amino acids. In some embodiments, pairs of amino acids are identified based on identifying pairs that the algorithm estimates have large degrees of variance in their distance measurements. For example, for a given protein sequence, the structure prediction algorithm is first performed to generate an in silico protein structure prediction and a distogram (probability distribution over distances between all pairs of residues). In some embodiments, a pair of amino acids is then identified if the two amino acids are separated on the linear chain by more than approximately five amino acids (i.e., more than five amino acids apart based on primary structure). In some embodiments, the pair of amino acids is identified based on having the distogram element with the highest variance. In some embodiments, the pair of amino acids is identified based on having a distogram element with one of the highest variances (e.g., 2^{nd}, 3^{rd}, 4^{th}, 5^{th}, 6^{th}, 7^{th}, 8^{th}, 9^{th}, or 10^{th} highest variance). Typically, k is between 1 and 100. The variance of a distogram element is a measure of the uncertainty provided by the algorithm about the distance between two amino acids. Selection is limited to only non-neighboring residue pairs because residues that are near each other on the linear chain are trivially close to each other in the physical structure.

In some embodiments, an algorithm-predicted factor is the relative importance of the distance between the two amino acids in the structure prediction algorithm (i.e., how important a particular distance is to the overall predicted structure). The importance of a particular distance relative to another depends on whether it is more or less likely to reduce the global uncertainty for the entire predicted protein structure. There are some distances between pairs of amino acids that are more critical for the algorithm to have as a constraint than others. This can be critical because some peripheral amino acid residues might have high variance or uncertainty in their measurement, but not be important for constraining the algorithm and the ultimately predicted structure. These peripheral amino acid residues might not have many interactions with other residues in the protein. Similarly, some pairs of amino acid residues might have low variance or uncertainty in their distance measurements, but they might be very important for constraining the algorithm and the ultimately predicted structure (e.g., due to their long-range interactions).

In some embodiments, an algorithm-predicted factor is the structural sensitivity of a pair of amino acids. Structural sensitivity may include whether that pair is involved in critical structural support (e.g. salt bridge, disulfide bond, key stabilizing interaction for secondary and/or tertiary structure). If the algorithm ranks a pair of amino acids as a sensitive location because it is critical that they be maintained, the algorithm is likely to de-emphasize the use of this pair for in vitro distance measurements. In contrast, amino acid pairs that that are not structurally sensitive (e.g., in loop regions, not part of a hydrogen bonding network in an alpha helix or beta sheet) would be prioritized by the algorithm for in vitro distance measurements. Structural sensitivity may include whether the amino acid pair is amenable to labeling with a FRET dye. For example, a solvent-exposed single cysteine that is not involved in a disulfide bond or a solvent-exposed lysine are ideal amino acids for labeling and would be ranked highly by the algorithm. In contrast, amino acid residues that would need to be replaced with artificial residues for labeling would be lowly ranked by the algorithm.

Second, the methods described herein involve measuring the distances between identified amino acid pairs in vitro using FRET, inputting those distance measurements into the algorithm to constrain the parameters of the algorithm (e.g., constraining the algorithm's output to agree with the measured distances), and determining, for a second time, a predicted structure of the protein using the refined structure prediction algorithm. From the biophysics of the FRET methodology, there will be an estimate for the uncertainty in distance measurement. The distogram output of the algorithm can be constrained such that the averages of the amino acid pair distances are the empirically FRET-measured values and the uncertainty of the amino acid pair distances are the standard deviations of the FRET-measured values. In some embodiments, this constraining of the algorithm is performed by setting the distributions of the FRET-measured values to be Gaussian with mean and standard deviation set as described above. With this new distogram, which is constrained to match the FRET-measured distances, the protein structure prediction algorithm may be run again to generate a more accurate and refined protein structure, starting with the distograms and angleograms.

### Whole-Genome Metagenomic Sequence Read Archives

In contrast to the large curated protein databases, which contain ˜200 million protein sequences, metagenomic sequencing read archives are among the world's largest databases of biomolecular sequences. For example, the NCBI sequencing read archive (SRA) contains more than 10¹⁶ bp of sequence data and is growing exponentially. Although organizations and tools such as MGnify assemble whole-genome metagenomic datasets from read archives into contigs/whole genomes, annotate predicted protein-coding sequences, and deposit those annotated sequences into curated databases, Applicants have noted that there can be a significant time-lag from when raw nucleic acid sequencing reads are deposited in a sequencing read archive to the submission to a curated database of the protein sequences predicted to be encoded by the genomes represented within, and some raw sequencing reads will never be assembled and curated at all (either because an entire dataset is not assembled/curated, or because some reads within an assembled dataset cannot be placed into sufficiently large contigs).

Although the SRA represents the richest, most up-to-date collection of the world's known genomic/metagenomic sequences, the publicly-available whole-genome metagenomic fraction of the archive includes well over 100,000 individual SRA “runs”, each of which contains unassembled, unannotated sequencing reads from an individual sequencing experiment run. As of 2019, the publicly-available whole-genome metagenomic fraction of the SRA contains ˜2×10¹² reads across >110,000 runs. In this format, the SRA cannot be directly searched by the typical MSA generation tools such as HHBlits and PSI-BLAST. One computational approach, “searchsra” (searchsra.org) can be used to search a fixed sample of nucleic acid sequencing reads from each of the totality of runs in the whole-genome metagenomic fraction of the SRA for nucleic acid sequences homologous (on the nucleic acid or protein level) to a search query.

The SRA, despite its massive size and utility for protein structure prediction, still contains only a tiny fraction of the total number of protein sequences that exist on Earth. Applicants have recognized that there remains an opportunity to mine additional protein-coding sequences directly from new, physical DNA samples that have yet to be sequenced and deposited in any form to a sequence database. However, standard DNA sequencing efforts to mine homologs from diverse DNA samples are unlikely to be the solution, as next-generation sequencing (NGS) technologies permit massively parallel sequencing of DNA but generate a finite number of reads per sequencing run. While abundant sequences in a given sample are readily detected with high confidence by modern NGS methods, Applicants have appreciated that rare sequences of interest, such as sequences coding for proteins homologous to a protein of interest, may not be sequenced deeply enough, even after multiple runs, to be detectable.

Target Enrichment

Target enrichment sequencing is one approach that can allow for confident base-calling for rare sequences. By enriching a complex sample for a specific gene or region of interest prior to sequencing, a researcher may largely eliminate off-target sequences and thereby only dedicate sequencing reads to genomic regions of interest. Applicants have appreciated that target enrichment can therefore enable the same number of reads to be devoted to a rare region/gene of interest as would require many standard sequencing runs on non-enriched samples, resulting in time and cost savings for homolog discovery.

There are several approaches that enable target enrichment sequencing. The simplest approach is to pre-enrich genomic regions of interest from a complex sample by amplification prior to sequencing, known as amplicon-seq (using, e.g., ILLUMINA® next generation sequencing (NGS) platforms). Primers designed to bind to a target nucleic acid sequence may be used to amplify homologous sequences from a complex mixture, where the nucleic acid sequence between the primer binding sites can diverge from known target-like sequences. However, as Applicants have appreciated, most amplification strategies are not tolerant of mismatches in the primer binding regions themselves. Therefore, amplicon-sequencing is somewhat limited in its ability to enrich homologs that are highly divergent in the primer binding regions. Amplification of full-length homologous genes is therefore especially problematic, as the terminal and flanking regions of genes are unlikely to be well-conserved. Furthermore, exponential amplification approaches can be challenging for nucleic acid targets that are present in very low abundance, since any low abundance nucleic acid not amplified in the first few rounds of amplification are unlikely to be detected at the completion of the reaction. Furthermore, amplification is difficult to multiplex and introduces sequencing errors that can complicate the identification of enriched variants that are truly sequence-divergent from the known target sequence(s).

Alternatively, target enrichment can be performed by nucleic acid hybridization capture. Because similar protein sequences are encoded by similar nucleic acids, and because similar nucleic acids have greater hybridization binding energy than dissimilar nucleic acids due to base pair complementarity, one can use nucleic acid binding assays to isolate nucleic acids from a complex mixture that resemble a given target sequence. There are a number of methods for nucleic acid hybridization capture by target sequence “probes,” including hybridization of complex mixtures to microarrays and to long single-stranded biotinylated oligonucleotide probes, immobilized on magnetic streptavidin beads. What is common to all of these strategies is that after an incubation period during which targets hybridize to the probes, repeated washes remove unbound, off-target sequences, while enriched homologous targets are retained on the immobilized probes. These hybridization-based approaches are more tolerant of mismatches than amplification based enrichment and avoid amplification bias, but they do select for sequences that have low rates of dissociation; if a candidate target dissociates from an immobilized probe during washing, it is removed from the reaction and can no longer be enriched, resulting in the discovery of only those homologs that rarely dissociate from the probes.

There is another hybridization-based technique, known as SCODAphoresis, that may be used to pre-enrich a sample for rare nucleic acids, making the subsequent sequence analysis of those nucleic acids far more effective. SCODAphoresis involves (i) loading a nucleic acid sample on a separation medium containing an immobilized probe, (ii) enriching the sample for nucleic acids complementary to the immobilized probe by applying a time-varying driving field and time-varying mobility field to the separation medium, and (iii) characterizing the enriched nucleic acid in the sample, including by sequencing. See, e.g., U.S. Pat. Nos. 9,512,477 and 9,534,304, incorporated herein by reference.

To date, for all of these approaches, target-enrichment sequencing has mostly been applied for the purpose of enriching clinical and/or human genomic samples for genes or panels of genes of interest. Herein, pre-enrichment allows for the devotion of fewer sequencing reads to a sample containing a single gene or collection of genes (e.g., cancer panel, or human exome) while maintaining high coverage. This results in cost and time savings. High read coverage is often used to allow for better gene variant determination, especially for the purposes of characterizing rare, disease causing genetic variants. Target enrichment has found ready application for single nucleotide polymorphisms (SNPs), insertion/deletion (indel) deletion, copy number variation (CNV) detection, and structural variation detection.

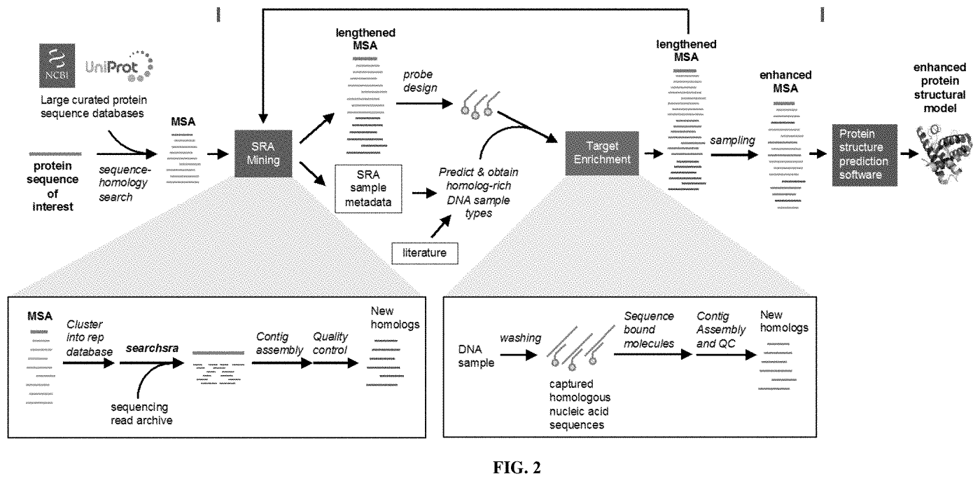

The present disclosure provides, in some embodiments, methods that use hybridization capture-based target enrichment for the intentional mining of highly divergent homologs (rather than more closely related/similar homologs) for a known protein to enhance structural prediction. FIG. 2 is a flow diagram of the steps of an illustrative process for discovering protein homologs, such as divergent protein homologs, which may include in silico homolog mining from metagenomic sequencing read databases and target enrichment. The methods provided herein, in some embodiments, are used for building an improved MSA for protein structure prediction that is larger and more diverse than MSAs compiled to date. This improved MSA can be used to generate higher quality DCA outputs, for example, which can be used in turn to train higher quality protein structure prediction models and execute higher quality de novo protein structure prediction.

In some embodiments, a method of the present disclosure comprises the following steps:
1. generating an initial homolog list for protein/protein family of interest by a sequence-homology search (pairwise or profile HMM-based; pre-computed or not) of one or more protein sequence databases;
2. from the initial list, generating a representative database (DBrep) of homologs related to the protein of interest (includes optional quality-control steps);
3. aligning the DBrep to a relatively small sampling of reads/read-pairs (e.g., 100,000) from every “whole-genome metagenomic” run in the SRA using searchsra.org;
4. ranking datasets prior to downloading to determine which are most likely to contain the most true homologs; ranking features can include (before/after false-positive removal):
   a. number of reads/read pairs in the 100,000-read sample giving an alignment probability value with DBrep above a certain threshold (“hit reads”);
   b. diversity of hit reads from the 100,000-read sample;
   c. total number of reads in the run;
   d. average length of reads;
   e. average length of hit read alignments;
   f. sequencing platform used; and
   g. Rread format (eg. paired or un-paired);
5. retrieving all reads from each “hit” (highly-ranked) SRA run;
6. optionally performing quality control steps to clean up unassembled reads from each “hit” SRA run;
7. aligning the DBrep protein list (with e.g., DIAMOND (Buchfink et al., Nat Methods 2015; 12: 59-60) or AC-DIAMOND or profile (with e.g., HMMSEARCH (Eddy et al. PLoS Computational Biology 2011; 7(10):e1002195)) to all nucleic acid reads/read-pairs or translated reads/read-pairs from every “hit” SRA run;
8. for each “hit” SRA run, assembling all full-length nucleic acid reads/read-pairs aligning to DBrep into contigs, using a fast assembler appropriate for the run's read format (paired/unpaired) and length (e.g., IDBA-UD (Peng et al., Bioinformatics 2012; 28(11): 1420-1428) for short reads);
9. translating open reading frames (ORFs) (e.g., all six possible ORFs) from assembled contigs to generate candidate protein homologs;
10. optionally performing quality control steps to validate candidate protein homologs as true homologs;
11. adding new homologs to the initial homolog list;
12. generating a new representative multiple sequence alignment (MSA) that has optimal balance of size and sequence diversity for DCA; and
13. performing feature extraction using the new MSC for co-evolution-based protein structure prediction model.

It is envisioned that at least some of these steps can be implemented by a processor such as that included in a computer (e.g., a general-purpose computer).

### An in Silico Multiple Sequence Alignment Method for Use in Co-Evolution-Based Protein Structure Prediction

There are trillions of sequencing reads/read pairs in the “whole-genome metagenomic” fraction of the NCBI Sequencing Read Archive (SRA) and additional sequencing reads in other metagenomic read archives (e.g. MG-RAST), and Applicant have appreciated that only a fraction of which have been assembled into contigs, annotated, undergone coding sequence translation and deposited into the large, curated NCBI and/or uniprot protein databases (200+ million protein sequences). In particular, metagenomic samples may include DNA from a multitude of organisms, spanning multiple kingdoms of life, including those that have never been previously identified, cultured or sequenced and thus contain highly diverse sequencing reads. Applicants have therefore recognized that metagenomic datasets represent a trove of additional protein sequences, from which homologs of a protein of interest may be identified.

A general illustrative method for in silico mining for new protein homologs includes the following steps.
1. Identifying a protein of interest for which a 3D structure is to be predicted.
2. Building an initial protein homolog sequence list, DBinit, for the protein of interest. This can be achieved by a number of means, including, for example:
   a. Searching protein family databases (e.g., InterPro, Pfam, CDD) for all proteins containing a given protein domain (architecture).
   b. Searching the NCBI non-redundant and/or uniprot protein sequence databases using pairwise (eg. BLAST, DIAMOND, AC-DIAMOND, PSI-BLAST), or profile HMM-based (eg. HHblits, JACKHMMER) alignment.
3. Optional: Assessing the completeness of the initial homolog list by downloading the entire NCBI non-redundant (nr) protein reference database and using it as a query against the DBinit initial database using DIAMOND, a fast and sensitive protein alignment tool adapted for large query sets, to search it for additional hits.
   a. To eliminate false-positive hits from this NCBI non-redundant search, the “Blast Score Ratio (BSR)” normalization method as described by Rasko et al. BMC Bioinformatics (2005) can be implemented, where the BLAST score for each non-redundant query hit against DBinit is normalized by its maximum possible score (a self-hit).
   b. Appending all true positives to DBinit.
4. Generating a representative reference database (DBrep) for all members of the protein family of interest by eliminating the presence of multiple sequences in DBinit that are very close in amino acid sequence space to each other. One non-limiting approach for doing this is to cluster DBinit by amino acid percent identity. For example, generate DBrep by clustering DBinit at, e.g., 90% using UCLUST.
5. Screening the SRA with the DBrep query using the public searchsra.org service to sample 100,000 reads from each of the “whole-genome metagenomic” runs in the SRA, likely revealing read hits over multiple individual SRA runs. Note that 100,000 reads is typically ˜1% of the complete dataset for any given SRA run, and thus represents a small fraction of the total reads.
6. Ranking datasets prior to downloading to determine which are most likely to contain the most true homologs. Ranking features can include (before/after false-positive removal):
   a. number of reads/read pairs in the 100,000-read sample giving an alignment probability value with DBrep above a certain threshold (“hit reads”);
   b. diversity of hit reads from the 100,000-read sample;
   c. totaling number of reads in the run;
   d. averaging length of reads;
   e. averaging length of hit read alignments;
   f. sequencing platform used; and
   g. reading format (eg. paired or un-paired).
7. Downloading the complete SRA run (all reads, not just a 100,000-read sampling) for any SRA runs that had positive hits in the 100,000-read sample OR a subset of those runs, for example, as triaged by the above ranking system, such that there is a minimum threshold rank to warrant downloading. Full SRA datasets are needed to search the entirety of the runs for additional reads that align to DBrep, to obtain high enough coverage of those genomic regions to be able to stitch shorter reads together into contigs that cover the full length of the protein of interest. Downloading can be performed using a number of approaches, including:
   a. manually downloading of individual SRA runs of interest;
   b. using commercial Aspera software, optimizing for efficient file transfer; and
   c. implementing a cloud transfer protocol to access SRA data in AWS (Amazon Web Service) or GCP (Google Cloud Computing) servers. This would allow for rapid, automatic execution of the pipeline and is the most robust option.
8. For each of the downloaded SRA run datasets, using an alignment tool to align all reads to the DBrep reference database. Multiple alignment tools could be used, including DIAMOND and HMMSEARCH (which requires translation first).
   a. Optional: Prior to contig assembly, aggregate reads from runs with the same sample origin to improve coverage.
9. For each dataset, assembling all hit reads into contigs. Multiple assemblers could be used, including:
   a. iterative de Bruijn Graph Assembler optimized for metagenomic data (IDBA-UD);
   b. a collection of different assemblers to be used across different SRA runs, where a strategy is used to identify the most optimal assembler for a given SRA run according to its unique read characteristics (e.g., read length, read format, coverage, etc); and/or
   c. de novo or reference-guided assemblers.
   d. Optional: Prior to assembly, false-positive hit read removal may be performed.
10. Open Reading Frames (ORFs) resulting in protein sequences greater than a cutoff fraction (e.g., 0.5-1.0, e.g., 0.7) of the length of the average DBrep protein member are then translated from these contigs in (e.g., all six (6)) reading-frames.
11. Translated ORFs in (e.g., all six (6)) reading-frames can be directly aligned (protein-protein) to DBrep to identify protein sequences aligning over a cutoff fraction (e.g., 0.5-1.0, e.g., 0.7) of the length of a DBrep member sequence.
12. Optional: Additional quality control steps may be performed, including of the following steps:
   a. detecting and remove artificial chimeras;
   b. aligning putative new homologs to all known protein sequences in a protein sequence database (e.g. NCBI nr) and the initial full database (DBinit); and
   c. if alignment to DBinit is better than to any non-DBinit member from NCBI nr, then putative homolog is considered a true homolog; and
13. Adding new homolog protein sequences to DBinit, generating an enhanced homolog listing, or DBenhanced.

It also is envisioned that at least some of these steps can be implemented by a processor such as that included in a computer (e.g., a general purpose computer).

### A Target Enrichment Sequencing Method for Enhancing a Multiple Sequence Alignment for Use in Co-Evolution Based Protein Structure Prediction

Protein coding DNA sequences from only a small percentage of life on Earth have been extracted, sequenced, annotated, and deposited into curated protein sequence databases. Target enrichment directly from previously uncharacterized DNA samples, including metagenomic samples, for the identification of new protein homologs is therefore especially advantageous for expanding the size and diversity of the list of known homologs of a protein of interest.

In some embodiments, a method of the present disclosure comprises the following steps:
1. generating an initial MSA for protein/protein family of interest by a sequence-homology search (pairwise or profile HMM-based; pre-computed or not) of one or more protein sequence databases;
2. from the initial MSA, designing one or more probes (e.g., nucleic acid, e.g., DNA, probes) that can hybridize to nucleic acid sequences that broadly represent the protein homolog family of interest;
3. immobilizing probes on a solid substrate, which could include a separation medium;
4. contacting probes with physical, complex DNA sample;
5. enriching homologs from non-homologs by selectively removing DNA unbound to the probes;
6. releasing bound homologs from the probes and sequence the DNA;
7. performing quality control steps to clean up sequencing reads;
8. aligning reads to the initial MSA used for probe design and if reads are shorter than the length of the full-length target sequence, assemble reads that positively align into contigs;
9. translating ORFs from aligned contigs to generate candidate protein homologs;
10. performing quality control steps to validate candidate protein homologs as true homologs;
11. adding new homologs to the MSA;
12. generate subset of the total MSA that has optimal balance of size and sequence diversity for DCA; and
13. performing feature extraction for co-evolution based protein structure prediction model.

One skilled in the art understands that there are multiple target enrichment strategies that may be employed. SCODAphoresis, for example, may be used for mining homologs from physical samples. In some embodiments, SCODAphoresis is used to purify divergent homologs from whole samples, where probes and target enrichment conditions are designed to enrich as many sequence variants as possible with relaxed stringency.

It also is envisioned that at least some of these steps can be implemented by a processor such as that included in a computer (e.g., a general purpose computer).

Probe Design

In some embodiments, designing a probe comprises the following steps.
1. Identifying a protein of interest for which a 3D structure is to be predicted.
2. Building an initial protein homolog sequence list, DBinit, for the protein of interest. This can be achieved by a number of means, including:
   a. searching protein family databases (eg. InterPro, Pfam, CDD) for all proteins containing a given protein domain (architecture); and
   b. searching the NCBI non-redundant and/or uniprot protein sequence databases using pairwise (eg. BLAST, DIAMOND, AC-DIAMOND, PSI-BLAST), or profile HMM-based (eg. HHblits, JACKHMMER) alignment.
3. Optional: assessing the completeness of the initial homolog list by downloading the entire NCBI non-redundant protein reference database and using it as a query against the DBinit initial database using DIAMOND, a fast and sensitive protein alignment tool adapted for large query sets, to search it for additional hits.
   a. To eliminate false-positive hits from this NCBI non-redundant search, implementing the “Blast Score Ratio (BSR)” normalization method as described by Rasko et al (2005), where the BLAST score for each non-redundant query hit against DBinit is normalized by its maximum possible score (a self-hit);
   b. Appending all true positives to DBinit.
4. Retrieving associated nucleic acid sequences associated with each protein record.
5. Generating an MSA for all members of the protein family of interest at the nucleotide level.
6. Generating a representative MSA (MSAref) by eliminating the presence of multiple sequences in MSA initial that are very close in sequence space to each other.
   a. One approach (among others) for doing this is to cluster MSA initial by percent identity. For example, generate MSAref by clustering MSA initial at 90% using UCLUST.
7. From MSAref, calculating the associated position-specific weight matrix (PWM). The PWM calculates both total information content and the weighted probability of finding any given nucleotide base for each individual position in the alignment.
8. Designing an optimal set of “probe” sequences most likely to hybridize to newly found homologs by:
   a. scanning through a sliding window of the MSA for different possible probe lengths;
   b. for each candidate probe (window of the MSA), calculating a probe score, comprised of the following metrics:
      i. mean information content (IC) from PWM;
      ii. longest sub-stretch of high IC bases;
      iii. percentage of low IC (degenerate) bases;
      iv. GC content (weighted by PWM);
      v. self-dimerization energy of consensus sequence; and/or
      vi. hairpin formation energy of consensus sequence;
   c. ranking probes by score and remove overlapping probes according to probe score, keeping the set of the most highly ranked, non-overlapping probes; and
   d. determining the optimal set of the most highly ranked, non-overlapping probes, with the lowest hetero-dimerization potential.
      i. One approach is to begin with the most highly ranked probe and calculate the hetero-dimerization potential for adding the 2^{nd} most highly ranked probe. If this passes an energy threshold, then add the 3^{rd} most highly ranked probe and repeat. If the 2^{nd} most highly ranked probe does not pass, move onto the 3^{rd} most highly ranked probe. Continue until the energy threshold can no longer be met.
9. Features of designed probes that are important for homolog mining:
   a. Probes can include non-standard nucleotide bases.
      i. Probes can include mixed/degenerate bases to increase the diversity of nucleic acid sequences that can be strongly bound/hybridized.
      ii. Probes can include locked nucleic acids and peptide nucleic acids to increase the melting temperature of a probe-target hybridization event.
      iii. Probes can include “universal” bases that base-pairing to multiple nucleotide bases, including 5′-nitroindoles and deoxylnosine bases, to increase the diversity of nucleic acids that can be strongly bound/hybridized.
   b. Optional: Simultaneously immobilize multiple probes for multiplexed target capture.
      i. Non-overlapping probes that tile the length of a target sequence can be immobilized in a single gel to increase the diversity of nucleic acid enrichment—so long as a target hybridizes to one probe it can be enriched, even if its sequence is divergent at the other probe sites.
      ii. Simultaneously enrich for multiple targets.
   c. Probes can hybridize nucleic acid targets anywhere along the sequence—in the middle or at the ends (unlike PCR based enrichment that requires the binding of two probes at opposite ends of a target molecule).
      i. Longer probes increase the diversity of nucleic acid enrichment by permitting hybridization to molecules that align at a minimum to a subsequence within the long probe.

It also is envisioned that at least some of these steps can be implemented by a processor such as that included in a computer (e.g., a general purpose computer).

Method for Fragmenting DNA Sample

The following is one example of a method for fragmenting a DNA sample.
1. Obtain whole samples from which new homologs are to be enriched. The following are features of nucleic acid containing samples that are important for target enrichment.
   a. Mobile samples can be complex, containing mixtures of nucleic acids with varying sequence homology to the probe set and non-nucleic acid molecules.
      i. Individual nucleic variants with high homology to the nucleic probe set can be extremely rare in the original sample.
      ii. Enrichment can be performed with metagenomic samples extracted from the environment that contain unknown mixtures of molecules, some of which have never previously been characterized.
      iii. Enrichment can be performed with samples isolated from one or more known organisms.
   b. Enriched nucleic acids can be linear or circular DNA molecules.
   c. Enriched nucleic acids can be single stranded or intact duplex DNA molecules.
      1. Can be fragmented by transposase.
      2. Can be fragmented by mechanical shearing.
      3. For example, can be fragmented to <3 kb for use with acrydite modified oligonucleotides immobilized in an acrylamide gel.
   d. Enrichment can be visualized and quantified by the incorporation of fluorescent dyes into the nucleic acid molecules undergoing enrichment.
2. Extract DNA from the sample using the appropriate method according to the sample type.
3. Optional: Samples that contain high molecular weight DNA can be fragmented prior to target enrichment. For SCODAphoresis, this would mean generating 1-3 kb fragments to facilitate electrophoretic mobility of the sample in the separation medium. Fragments may be generated by:
   a. physical DNA fragmentation (e.g. sonication, shearing);
   b. chemical fragmentation; and/or
   c. enzymatic fragmentation (e.g., nuclease, transposase treatment).
4. Ligate adapter sequences to the 3′ and 5′ ends of the fragmented DNA molecules to be used as PCR primer handles downstream.
5. In one implementation, fragmentation and adapter ligation are combined in a single transposase mediated step:
   a. assemble transposomes consisting of annealed adapter oligos and MBP-tagged Tn5 transposase enzyme (transposomes may be used fresh, or stored frozen);
   b. prepare reaction with transposomes and DNA at 10:1 Tn5:DNA mass ratio; incubate at 55° C. for 80 minutes;
   c. stop fragmentation and adapter addition (aka “tagmentation”) reaction by adding 0.2% SDS and incubating at 55° C. for 10 min;
   d. clean up DNA reaction with size-selection using SPRI (e.g., AMPure) beads
6. Optional: To generate more adapter-appended, fragmented DNA, perform PCR amplification. To minimize PCR bias, chimeric product generation, and other errors during amplification:
   a. use 0.1 ng/uL DNA template (final concentration in the amplification reaction); and/or
   b. amplify for 12 cycles.

It also is envisioned that at least some of these steps can be implemented by a processor such as that included in a computer (e.g., a general purpose computer).

Method for Targeted Enrichment

The following is one illustrative example of a target enrichment process.
1. Flow complex DNA sample over immobilized probes in hybridization buffer.
2. Remove weakly or non-specifically hybridized “off-target” DNA molecules by repeated washing.
3. Release tightly, specifically hybridized “target” DNA molecules from the immobilized probes.

In some embodiments, SCODAphoresis is used for target enrichment of divergent homologs from a DNA sample. An instrument that can perform SCODAphoresis (i) contains multiple electrodes for generating dynamic electric fields (ii) Contains one or more temperature controllers for the uniform or non-uniform generation of temperature gradients in the electrophoresing gel (iii) incorporates sample inlet ports, enriched sample recovery port, outlet ports for highly mobile sequences.

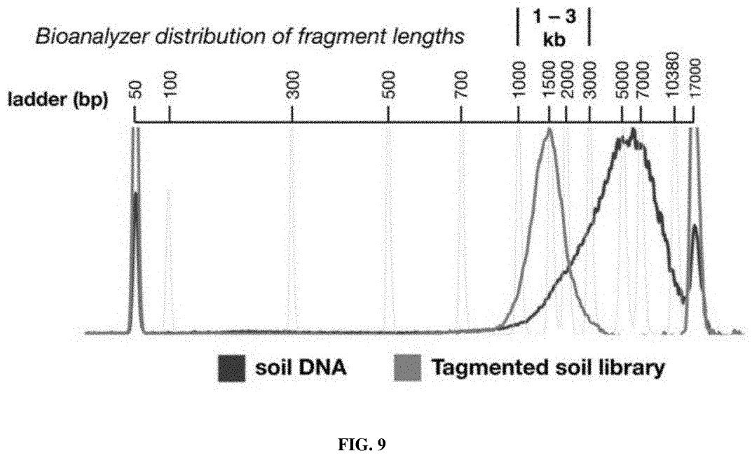

SCODAphoresis, in some embodiments, may include the following steps:
1. The separation of nucleic acid variants is achieved by repeated on/off binding interactions between nucleic acids and immobilized probes that results in a differential mobility for each individual nucleic acid variant.
2. The mobility of nucleic acids is driven by an electric field, resulting in electrophoresis of nucleic acid variants through gel-immobilized probes.
3. A user can remove higher mobility (less tightly bound) sequences by electrophoresing them away and thereby enrich the remaining (more tightly bound) sequences.
4. A nucleic acid can still be low mobility in the gel, but contain multiple mismatches to the probe—non perfect sequence complementarity.
5. Control over the stringency of the separation is tuned by temperature, the number of enrichment iterations, probe concentration, and probe design. See FIG. 10, which suggests that through interaction of all of these parameters, the stringency of enrichment of a sample can be tuned—where high stringency target enrichment purifies nucleic acids most homologous to the original target (Phi29) and more relaxed target enrichment purifies even divergent (40-50% homology) nucleic acids.

It also is envisioned that at least some of these steps can be implemented by a processor such as that included in a computer (e.g., a general purpose computer).

### An Iterative Homolog Discovery Method Including Synergistic in Silico and Physical Assays

In silico homolog discovery enables metagenomic sequencing reads collected from locations across Earth's biosphere to be screened broadly (but shallowly, since sequence reads were not pre-enriched) for homologs of a given target sequence. In the process, metagenomic archive mining gathers two useful pieces of information (1) an expanded set of homologs for probe design, and (2) from the sequencing read metadata, identification of which ecosystems or organisms were the richest in homologs, suggesting where to sample in the future. Hybridization capture target enrichment can then be applied to newly collected physical samples likely to be enriched for the protein family of interest, and then enrich it from homologous sequences thousands-millions times more, much like an oil-drill is applied after global screens. Once target enrichment reveals additional homologs, one can return to in silico homolog mining and search for further homologs from the expanded definition of the homolog family. Algorithms that work only on large curated protein sequence databases (such as PSI-BLAST and HHblits) use such an iterative strategy for extra-sensitive homology searches. The present disclosure provides, in some embodiments, an iterative strategy between in silico broad sequencing-read archive searches and physical, narrow target enrichment searches, creating a synergistic cycle between the two.

In some embodiments, a method of the present disclosure comprises the following steps:
1. generating an initial homolog list for protein/protein family of interest by a sequence-homology search (pairwise or profile HMM-based; pre-computed or not) of one or more protein sequence databases;
2. metagenomic sequence read homolog mining (see Example 1) broadly screens submitted metagenomic sequencing reads for new homologs;
3. based on the lengthened MSA (includes new homologs identified by in silico mining), designing “probes” for target nucleic acids;
4. downloading metadata for metagenomic samples with positive homolog identification to reveal the ideal sample collection type and location for the target protein family;
5. obtaining a physical DNA sample predicted to be rich with putative homologs;
6. performing hybridization-capture target enrichment with designed probes and chosen DNA sample (see above);
7. from target enrichment sequencing data, identifying new homologs;
8. generating lengthened MSA; and
9. with lengthened MSA, repeating steps 2-8 (repeating iteratively).

It also is envisioned that at least some of these steps can be implemented by a processor such as that included in a computer (e.g., a general purpose computer).

Direct Coupling Analysis

When generating contact map predictions, it is necessary to go beyond the raw correlations, due to the fact that some observed correlations may indirect. For example, if residue A interacts with residue B, and residue B interacts with residue C, there will be a substantial correlation between residues A and C, but no true contact between A and C. To leverage co-evolutionary data for accurate structural determination, it is necessary to distinguish direct and indirect correlations. The state-of-the-art algorithm for deducing direct correlations is called Direct Coupling Analysis (DCA). Once a collection of all the known protein sequences that are homologous to a protein of interest have been assembled into a multiple sequence alignment (MSA), direct coupling analysis (DCA) can be performed to solve a Potts model on the alignment. The output of (DCA) is a matrix that represents the “strength” of the coupling between all pairs of residues. Empirically, it has been demonstrated that a high DCA output value often indicates that the two residues are physically in contact. The quality of the DCA analysis is measured by the extent to which the output, when threshold appropriately, produces accurate predictions for whether or not each pair of residues is in contact (defined by being within a certain distance from each other). Using a predicted three-dimensional structure based on DCA, one can identify pairs of amino acids that have high variance in the spatial distance between the two amino acids. As described herein, researchers may then take these amino acids identified in silico and determine the experimental distance between them in vitro, e.g., in order to refine the DCA predictions and/or the protein structure prediction models.

Three-Dimensional Structure Prediction from DCA Generated Contact-Maps

Computer-implemented protein structure prediction models (e.g., neural network models) may be applied to predict the three-dimensional structure of the protein (e.g., a protein sequence obtained using Multiple Sequence Analysis (MSA)) from the contact maps generated by DCA. In some embodiments, a protein structure prediction model is AlphaFold, as developed by Google DeepMind.

In some embodiments, a protein structure prediction model comprises four primary steps:

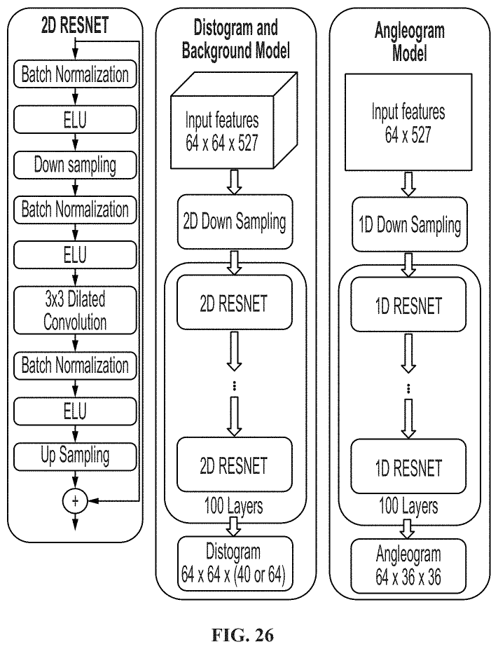

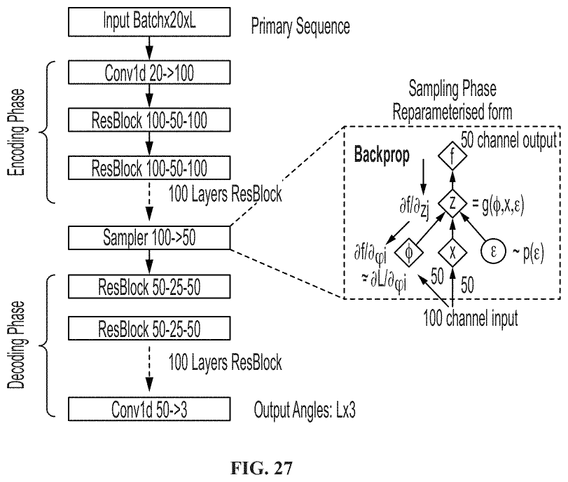

(1) Posterior distribution estimation. This is trained with full knowledge of the statistical features and amino acids of a multiple sequence alignment (MSA) of a target protein (shown as “distogram model” in FIG. 25). In some embodiments, the posterior estimator is a 2D Resnet, optionally with 220 layers, which is trained with a full set of input information (FIG. 26).

(2) Prior distribution estimation. These estimations are based on protein length and locations of Glycine amino acids (shown as “background model” in FIG. 25). The prior distribution estimation entails a similarly structured Resnet as the posterior distribution estimation but is trained on different input. (FIG. 26).

(3) Torsion angles distribution estimation. These estimations are used as initialization generative model in maximum likelihood (ML) estimation of protein structure (shown as “angleogram model” in FIG. 25). In some embodiments, the angleogram distribution estimator is a 1D Resnet which has a structure similar to the posterior estimations. The input is also similar to the inputs for the posterior estimations, but the output is the joint distribution over (Φ,Ψ,Ω) torsion angles. The initial angle estimation is important for the optimization process as the final folding model is highly dependent on it.

(4) Solving a maximum likelihood estimation by optimizing over two torsion angles. To perform maximum likelihood estimation over each protein structure (e.g., the distance matrix), a differentiable model from torsion angles to distance matrix is required. To reduce the complexity of this problem, it is assumed that the C—C and C—N bound lengths are fixed to a predefined value and the torsion angle is fixed to 180 degrees. In some embodiments, this step is implemented using Torch or Tensorflow. These functions are flexible to incorporate all bond lengths and torsion angles to the optimization process.

A protein structure prediction model may be implemented for protein structure prediction downstream of DCA-based feature extraction. In some embodiments, prior, posterior and angleogram models may be trained by applying random croppings of full pairwise features. These crops are designed to cover the full protein but with random onsets. This leads to a data augmentation process that prevents the model from over fitting and makes it robust to shifts in the peptide chain. To predict the 3-D structure of a protein, a multiple sequence alignment (MSA) is first performed for that protein, followed by feature extraction by computing Potts model parameter and applying DCA. The prior and posterior distograms are then obtained using these features. The likelihood function is then obtained by dividing the posterior estimations over the prior estimations. The final step of optimization is to perform a repeated gradient descent over the (Φ,Ψ,Ω) torsion angles.

### Generative Model and Directed Evolution of Protein Sequences

Generating new functional proteins, which exhibit increased function with respect to some desired activity, can be a fundamental step in engineering proteins for a variety of practical applications. The fitness of a protein with respect to a particular function may be closely related to the three-dimensional (3D) structure of that protein. Directed evolution is one process by which new functional proteins may be generated. In the context of functional protein generation, directed evolution may involve a repeated process of diversifying, selecting, and amplifying proteins over time. In general, such a process may begin with a diversified gene library, from which proteins may be expressed and then selected based on their fitness with respect to a desired function. The selected proteins may then be sequenced, and the corresponding genetic sequences amplified in order to be diversified for the next cycle of selection and amplification.

As proteins are repeatedly selected based on their fitness with respect to a desired function, increasingly fit protein variants are incrementally generated over time. Directed evolution may be thought of as traversing a local protein function fitness landscape, wherein the rounds of selection determine the most optimal gradient in the protein function fitness landscape given the starting point of the initial diversified gene library. Applicants have recognized and appreciated that having a better designed initial diversified gene library results in a better exploration of the protein function fitness landscape, thereby minimizing the number of rounds of evolution required to converge to an optimum and providing a resulting reduction of the cost and time associated with generating functional proteins. Thus, as described herein, designing initial diversified gene libraries with enhanced properties, such as increased diversity or greater initial protein function fitness, is advantageous for the directed evolution of functional proteins.

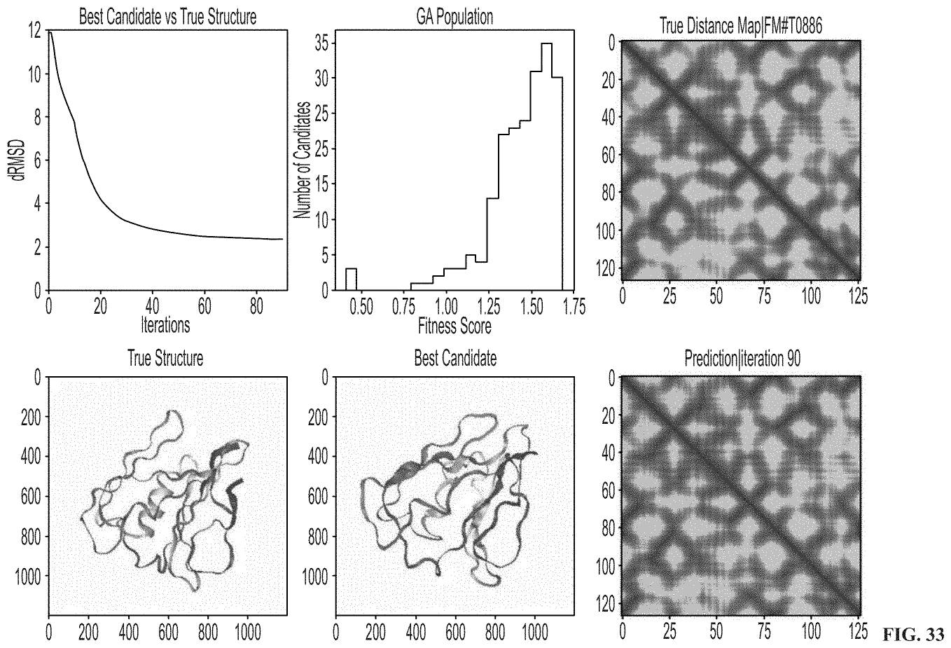

The present disclosure provides, according to some embodiments described herein, a generative machine learning model that generates new functional protein sequences given an input protein structure, yielding multiple candidate protein sequences that are diverse (e.g. different in sequence from known, natural protein sequences) yet are likely to retain a same or similar 3D structure to the input protein structure. FIG. 34 is flow diagram of an illustrative process for generating new functional protein sequences according to some of the techniques described herein. As shown in the illustrated example, the input protein structure may be an experimentally-derived (e.g. known) structure model. In other examples, the protein structure provided as input to a generative machine learning model may itself optionally be an output of an in silico protein structure prediction algorithm. In silico protein structure prediction algorithms may include, for example, homology modelling, modelling with machine learning, or alternative approaches, such as those described herein.

Regardless of how the input protein structure is derived, it may then serve as an input to generative machine learning model, as shown in the figure. In the illustrated example, the input protein structure is a backbone structure of the protein. The backbone structure of the protein may be indicative of the overall structure of the protein and may be represented as a list of Cartesian coordinates of protein backbone atoms (alpha-carbon, beta-carbon and N terminal) or a list of torsion angles of the protein backbone structure. Regardless of how the input protein structure is represented, the generative machine learning model may process the input protein structure in phases of encoding, sampling, and decoding, as indicated in the figure, and described in detail below, in order to produce as output new functional protein sequences.

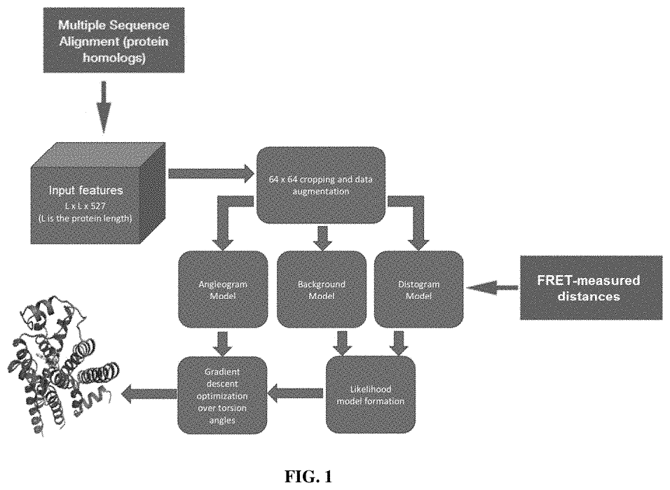

According to some embodiments, a generative machine learning model such as the one described with reference to FIG. 34 may be used alone, or iteratively in conjunction with an in silico protein structure prediction algorithm to allow for a closed-loop, machine-learning guided platform for directed evolution. FIGS. 1 and 25 are flow diagrams illustrative of such a closed-loop, machine-learning guided platform for directed evolution, such as may be used to design new functional protein sequences having enhanced or optimal fitness with respect to a desired function. As shown in the illustrated example, a directed evolution process using a generative machine learning model according to the techniques described herein may involve the following steps:

(i) an initial protein structure model is provided as the input protein structure to a generative machine learning model, such as described above;

(ii) the generative machine learning model generates new protein sequences predicted to fold into the input protein structure;

(iii) a diversified gene library is synthesized from the new protein sequences

(iv) optionally, the gene library may be further diversified, for example by mutagenesis or DNA shuffling or other suitable techniques;

(v) the diversified gene library is expressed;

(vi) high fitness proteins are selected from the expressed proteins;

(vii) the selected proteins are sequenced, and the genes coding for the selected proteins are amplified;

(viii) the amplified gene sequences are diversified for another cycle of selection and amplification. Diversification may be achieved by:

1. repeating steps (iv)-(vii).

2. the amplified gene sequences are fed into a protein structure prediction algorithm; and then steps (ii)-(vii) are repeated.

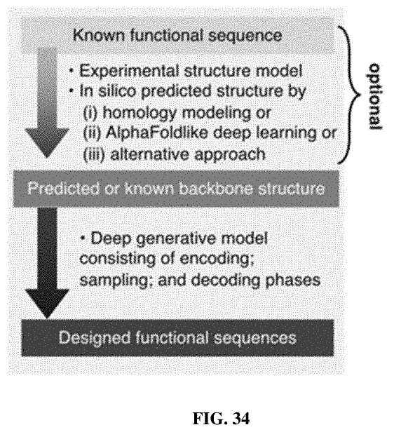

This completes the closed-loop cycle of directed evolution, which may be run iteratively as protein sequences converge on a functional protein sequence with optimal fitness with respect to a desired function. It should be appreciated that some steps of the process illustrated in FIG. 35 are optional and may be skipped or replaced with alternative steps in some embodiments. For example, the use of traditional diversification techniques in (iii) need not take place in every iteration and may not take place in any iterations. It should also be appreciated that the process illustrated in FIG. 35 need not repeat ad infinitum, but may instead terminate, such as when the protein sequences have converged on a functional protein sequence with a degree of fitness with respect to a desired function above a threshold.

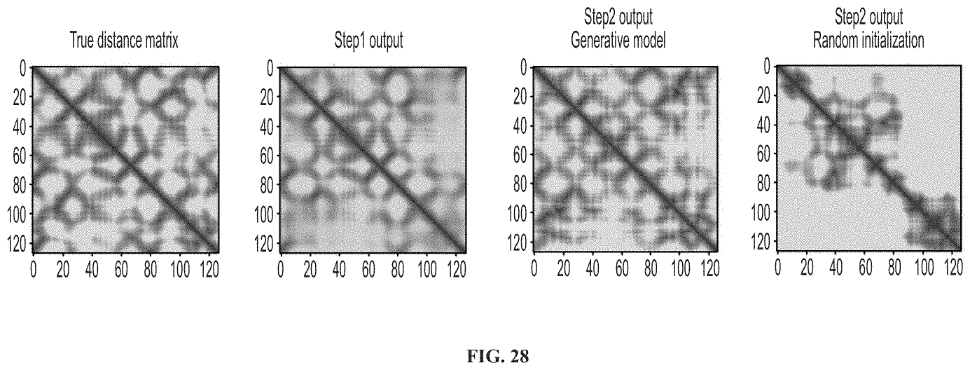

In the context of a closed-loop directed evolution cycle, as shown in FIG. 35, the generative machine learning model serves to produce a higher quality diversified gene library than may be obtained by random mutagenesis or other traditional techniques. Having learned the distribution of sequences that fold to structures similar to the input structure, as described in detail below, the generative machine learning model produces multiple candidate protein sequences for inclusion in the diversified gene library that are significantly more likely to fold and function similarly to, or better than, the original input sequence, when compared to candidates sequences obtained through random mutagenesis or other traditional techniques. Moreover, although the space of possible protein sequences of a given length is astronomically large, the generative machine learning model learns to only produce sequences that are likely to have a similar functionality and structure as a given target. In FIG. 27, a flow diagram illustrating an exemplary implementation of a generative machine learning model according to the techniques described herein is provided. In the illustrated example, the generative machine learning is implemented as a deep neural network comprising phases of encoding, sampling, and decoding. It should be appreciated that the deep neural network of FIG. 27 is exemplary, and that alternative machine learning methods and architectures may be employed in some embodiments of the techniques described herein.

### Generative Models Provide Good Structure Initializations

The maximum likelihood (ML) optimization surface is non-convex and will include many local minima and saddle points. To mitigate that issue, one may start the gradient descent from model-guided initial presumptions. Model-guided initial presumptions can be obtained by sampling a target protein's angleogram multiple times and/or by generating many samples using a variational encoder-decoder; and then computing a distance matrix for each initialization point. From this selection of initialization points, one can select the points with the highest structural scores.

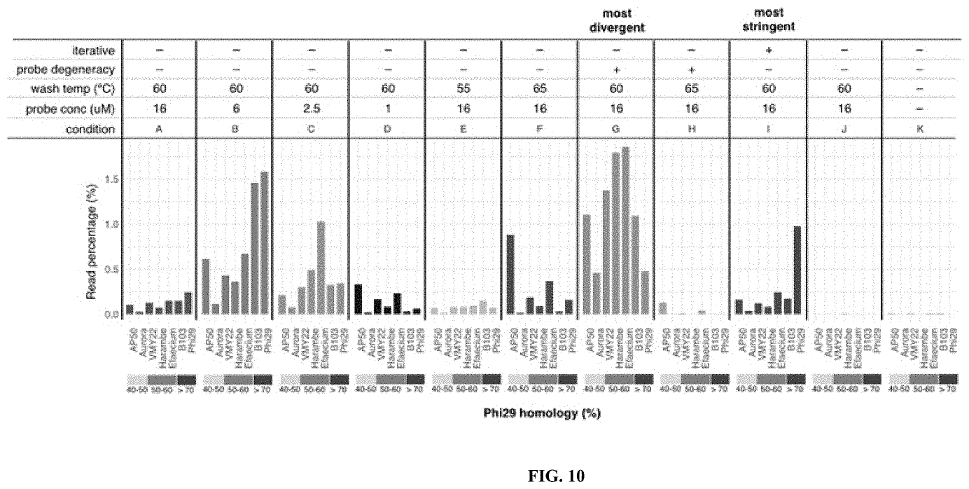

In order to obtain a good starting population of candidate protein structures, the inventors have developed a 1D deep resnet generative model (FIG. 10) from the primary sequence to protein structure, wherein each structure is represented by a sequence of triplet dihedral angles (Φ,Ψ,Ω). This generative model is designed to sample different possible structures, such that many candidate structures can be obtained from a single primary sequence. Initializing gradient descent with many candidate structures from a generative model improves the final model output, which is a distance matrix capturing the structure of the target protein, relative to random initialization (FIG. 11).

The 3-D backbone structure of a target protein could be represented by cartesian coordinates of protein backbone atoms (alpha-carbon, beta-carbon and N terminal) or by a list of torsion angles of the protein backbone structure. Because cartesian coordinates of protein backbone atoms can be directly converted to a sequence of triplet dihedral angles (Φ,Ψ,Ω), a “sequence to structure” model takes the primary sequence input as a list of one-hot vector(s) (20 dimension) and output structure(s) as a list of torsion angles. For a protein structure with L amino acid residues (L×20 matrix), the structure could be represented by a L×3 matrix (i.e., 3 torsion angles (Φ,Ψ,Ω)). This model, which comprises three discrete phases, is described in FIG. 10 and below:

(1) Encoding phase. The input layer is propagated through the Conv1D project (20 dimension to 100 dimensions), which generates a 100×L matrix. This matrix is iterated 100 times through a residual network (RESNET) block (Fig.ResBlock1D) that performs batch norming, applies the exponential linear unit (ELU) activation function, projects down to 50×L, applies again batch norming and ELU, and then cycles through 4 different dilation filters. The dilation filters have sizes 1, 2, 4, and 8 that are applied with a padding of the same to retain dimensionality. The final batch norm, the matrix is projected up to 100×L and an identity addition is performed.

(2) Sampling phase. A 100×L matrix is generated from the encoding phase, and the first 50 dimensions from the encode vector in each position serve as the mean of 50 gaussian distributions and last 50 dimensions serve as the log of variance of the corresponding gaussian distributions. After applying a reparameterization trick, the model samples the hidden variable z from 50 gaussian distribution, which together generates 50×L matrix as output.

(3) Decoding phase. The input for the decoding phase is the 50×L matrix output from the sampling phase, and it iterates a similar ResBlock as in the encoding phase for 100 times (The primary difference from the encoding phase ResBlock is that the ResBlock module of the decoding phase maps 50 dim to 50 dim input). After ResBlock layers, the model reshapes the 50 dimension to 3 dimension (corresponding to 3 torsion angles) using 1D convolution with kernel size 1.

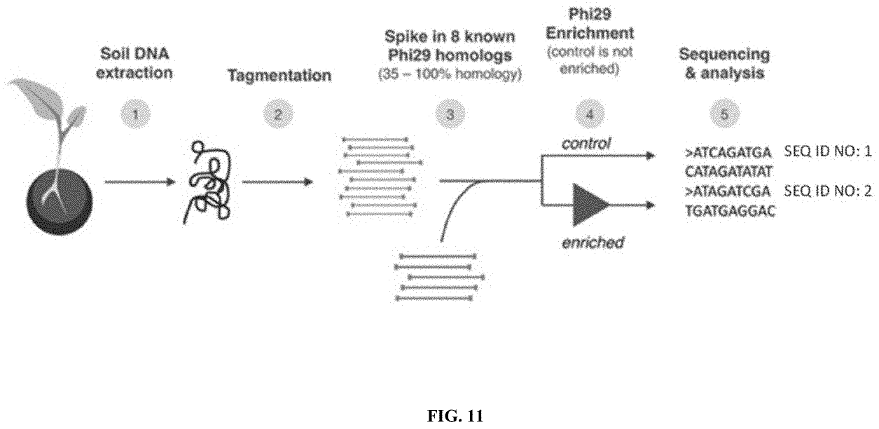

The initial starting point is important for gradient descent optimization. After experimenting with different global optimization approaches, it was found that a genetic algorithm (GA) with two specific mutation operation works well for structure prediction (FIG. 12)

Given a primary sequence, the generative model described above may be used to generate 200 candidate structures as an initial population. Each structure may be represented by a sequence of triplet dihedral angles (Φ,Ψ,Ω). Direct gradient-descent optimization for each structure in the 200 may be implemented. After at least 1,000 direct gradient-descent steps, the genetic algorithm (cross-over mutation within 200 population and randomly select position to flip the Omega angle) may be used as a new generation for direct optimization. After each round of GA interaction, one may keep the highest performer (without cross-over) in the new population.

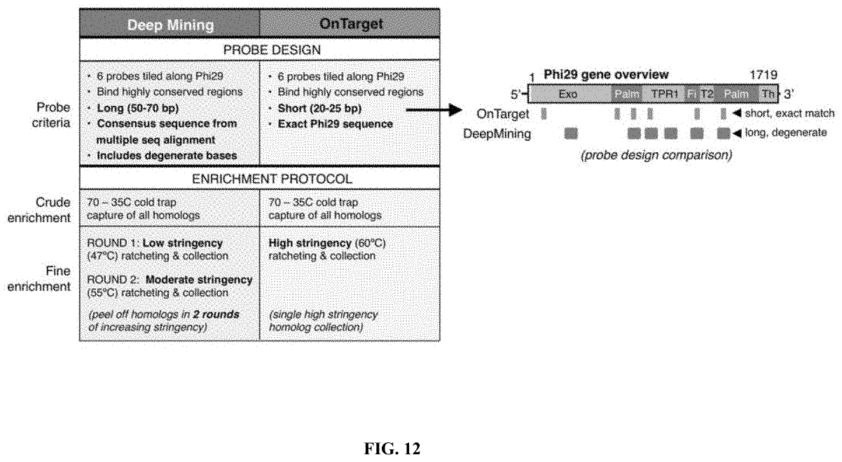

The inventors of the present disclosure have found that a protein structure prediction model such as AlphaFold, with 40 bins could learn a high-performing pair-wise distance matrix. In some embodiments, the the step 1 model may be re-trained to output 64 bins to cover distance range 0 Å to 32 Å (0.5 Å per bin). The 64-bin framework gives high resolution and reveals better local structure detail. See FIG. 13.

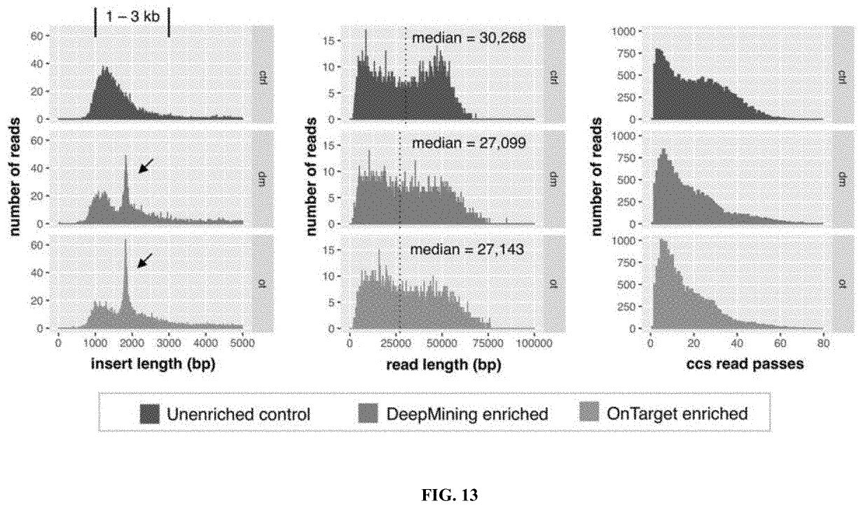

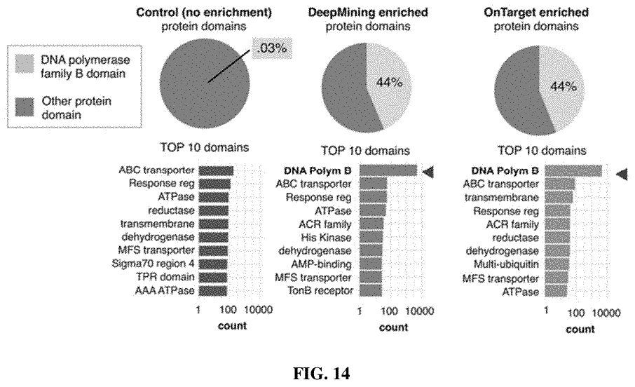

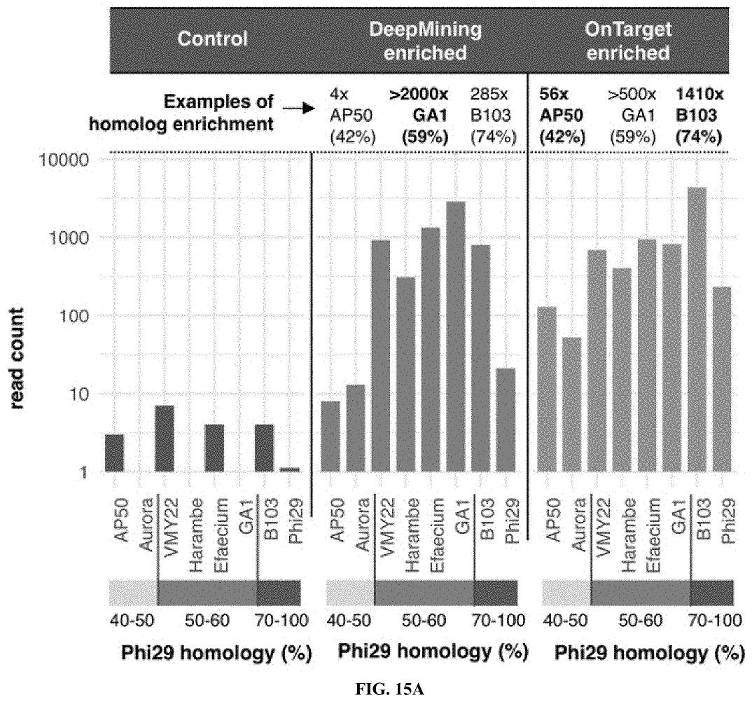

A set of evaluation/convert/plotting python scripts have been developed to allow for acquisition of a unique metric used (dissimilar from previously reported metrics) for ascertaining how well a model algorithm predicted a given protein's structure (FIG. 14). The evaluation framework also contains built-in visualization. (FIG. 15).

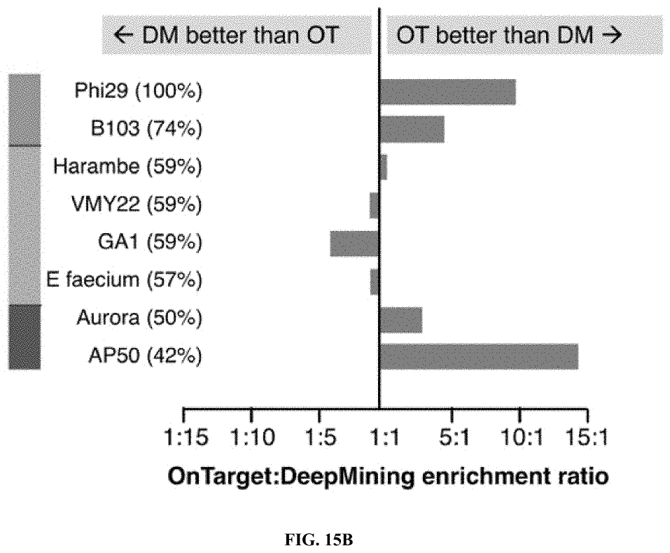

In some embodiments, a fully implemented in silico protein sequence to structure prediction has been performed. An example predicted structure versus the ground-truth structure is shown in FIG. 16.

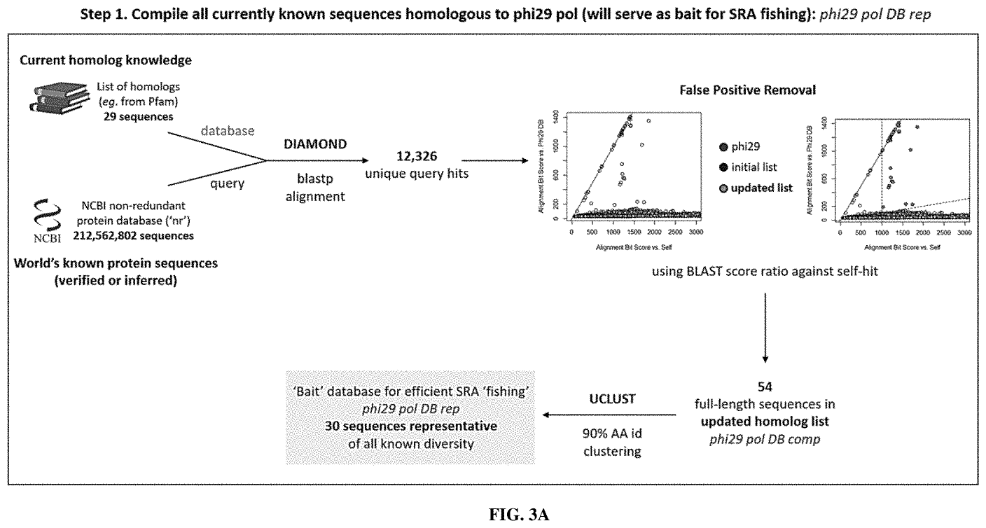

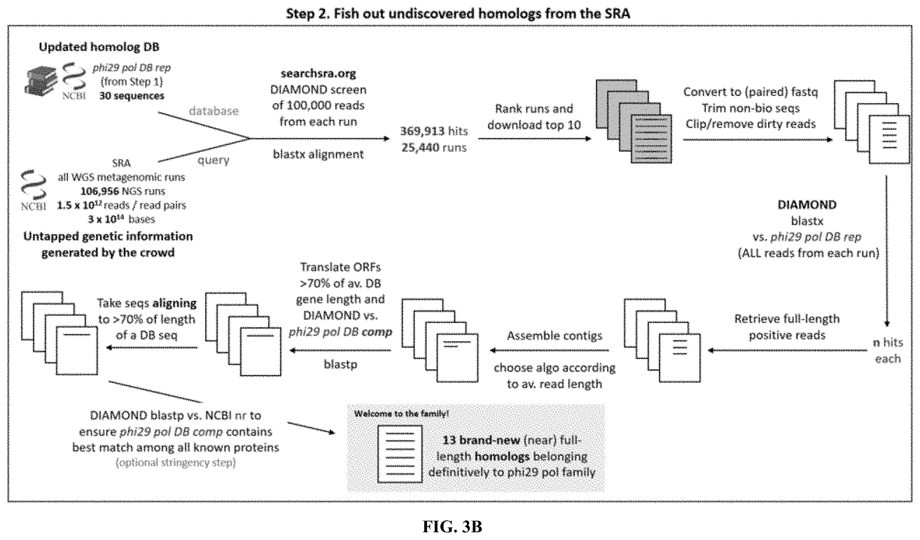

FIG. 4 is a flow diagram illustrating an exemplary ResBlock, according to some embodiments of the techniques described herein. As was described with reference to FIG. 3, this flow diagram indicates that a ResBlock may function according to the following steps:

(i) Applies batch normalizing (BatchNorm);

(ii) Applies the exponential linear unit (ELU) activation function;

(iii) Projects down to a 50×L matrix using a one-dimensional convolution (Conv1D);

(iv) Applies batch normalizing (BatchNorm) and ELU;

(v) Cycles through 4 different dilation filters (Dilated Conv1D), having sizes 1, 2, 4, and 8 a padding of the same to retain dimensionality;

(vi) Applies batch normalizing, projecting the matrix up to 100×L;

(vii) Performs an identity addition.

A deep neural network according to the techniques described herein, such as illustrated in FIGS. 3 and 4, for example, may be trained by providing training data to the network in pairs of input protein structures and corresponding target protein sequences. In order to learn a statistical model of the input distribution, an input protein structure may be provided as input to the deep neural network, which may output a protein sequence, such as by the process described with respect to FIGS. 3 and 4 above. A loss value may then be calculated between the neural network's output protein sequence, and the target protein sequence corresponding to the input protein structure. Then, a gradient descent optimization method can be applied to update weights or other parameters of the neural network such that the loss value is minimized.

As a specific example of training, such a deep neural network may be trained using existing protein/domain structure databases like PDB (Protein Data Bank) and CATH (Class, Architecture, Topology, Homologous superfamily), which contain both structure and primary sequence information. The information of given backbone structure may firstly be converted to a list of torsion angles. The list of torsion angles may be provided as input to the neural network, which may output a 20 dimension probability vector for each residue, representing the probability of 20 amino acid in each residue position. A cross-entropy loss may be computed between the output probability vectors and true primary sequence; then, any general stochastic gradient descent optimization method can be applied to update the model parameters and minimize the loss value.

It should be appreciated that any of the parameters of a deep neural network according to the techniques described herein may differ from those in the example of FIGS. 3 and 4. For example, in some embodiments, the dimensionality of the layers of the deep neural network may differ, or other parameters that may be associated with the network, such as type and number of activation functions, loss function, learning rate, optimization function, etc, may be adjusted. Moreover, the architecture of the deep neural network may differ in some embodiments. For example, differing layer types may be employed, and techniques such as layer dropout, pooling, or normalization may be applied.

With regards to the techniques described herein for generating new functional protein sequences, Applicants have further discovered and appreciated that in order to generate enhanced diversified gene libraries, it is not only important that functional protein sequences are generated that could fold into a given input protein structure (so as to retain some degree of function), but also that the generated functional protein sequences are diverse—that is, they are dissimilar to the set of known or naturally-occurring sequences associated with the input protein structure. New functional proteins generated in such a way are more likely to have new or enhanced function, relative to functional proteins generated by traditional methods, and thus provide an initial diversified gene library with increased diversity and protein function fitness.

According to some embodiments, new functional protein sequences that exhibit increased diversity with respect to an input protein structure may be generated by first determining a set of known protein sequences having a structure similar to the input protein structure, then repeatedly generating candidate functional protein sequences and discarding any that are determined to be too similar to members of the set of known protein sequences. As part of repeatedly generating candidate functional protein sequences, a generative machine learning model, such as according to the techniques described herein, may be employed.

As a specific example, new functional protein sequences that exhibit increased diversity may be produced by the following method:

1. Given an input protein structure (e.g. only consider the backbone), search all similar structures (e.g. could be domain structure) under certain similarity criteria (e.g. Root-mean-square deviation below a certain threshold, such as 2), and obtain the primary sequences for those similar structures as the set of known sequences that fold into those structures.

2. Use a generative model, such as one according to the techniques described herein, to generate new functional protein sequences from the given input structure. Accept the generated sequence only if the generated sequence is below a certain similarity threshold (e.g. identity percentage less than a threshold, such as 80%) to all the sequences in the set of known sequences. The generative model would stop once the number of accepted sequences reaches a specified value (e.g. specified by a user).

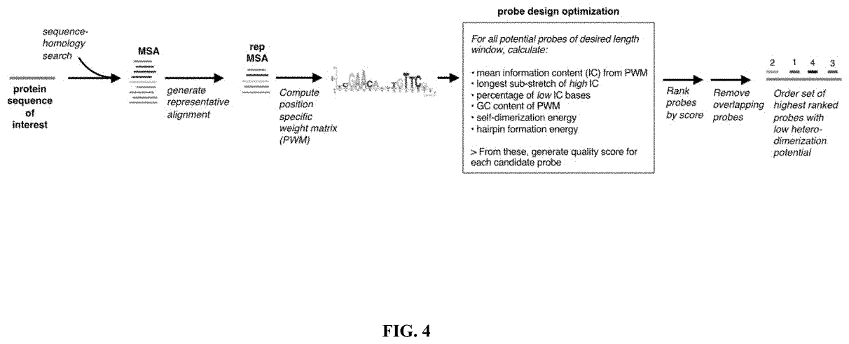

FIG. 5 is a sketch illustrating pseudo code for generating diverse (“low-identity”) functional protein sequences, according to some embodiments. As input, the pseudo code takes in a 3D Structure S (e.g. a protein structure, represented in any suitable way), a struct2seq model F (e.g. any suitable generative machine learning model), a requested number of candidate N (e.g. the desired number of new functional protein sequences), and an identity threshold k (e.g. an upper bound on the allowable similarity between a generated functional protein sequence, and known sequences). As described above, the pseudo code then enters a loop wherein a final candidate set is populated by means of repeatedly: proposing a candidate sequence x using F(S); checking if x is similar to known sequences under k; skipping x if so, and adding x to the final candidate set otherwise. This process is repeated until the size of the final candidate set is equal to N, at which point the process ends.

### Identification of Sites for Measurement

Identifying a pair of two amino acids that should be labeled for determination of the distance between them can be a challenging problem for several reasons. First, for an average protein comprising a length of 500 amino acids, empirically measuring the distance between every pair of amino acids in vitro would be impractical (protein of 500 amino acids has ˜125,000 pairs of amino acids). Second, many of the amino acids of a given protein (e.g., glycine residues) are not amenable to labeling with fluorescent dyes and swapping these amino acids for ones that could be labeled would have a high probability of destabilizing the protein structure. Therefore, care must be taken to pick residues that are least likely to disrupt the protein structure and that will maximally improve the accuracy and usefulness of the structure model of the protein of interest. Furthermore, a maximum of two available labeling sites should be chosen for each protein variant, ideally wherein each amino acid site for labeling is an estimated 2-10 nanometers from one another. In some embodiments, the two amino acids in a pair of amino acid residues in a protein are estimated to be about 2, 3, 4, 5, 6, 7, 8, 9, 10, 11, 12, 13, 14, 15, 16, 17, 18, 19, or 20 nanometers apart from one another.

In some embodiment, labeling is done at two solvent-accessible cysteines or lysines or a combination of the two that are within 10 nanometers but may or may not be forming disulfide bonds with each other. In one embodiment, all of the native cysteines but one or two are replaced with other amino acids that cannot be labeled. Cysteines that form disulfide bonds with other cysteine may not be necessary to get rid of as they are likely locked into their disulfide bonds and serve an important stabilizing function for the protein structure and furthermore may be nonreactive with FRET dyes.

In some embodiment, the two amino acids of a pair are solvent-exposed (or solvent-accessible). In some embodiments, at least one of the two amino acids of a pair is a solvent-exposed essential amino acid. In some embodiments, at least one of the two amino acids of a pair is a naturally-occurring amino acid. In some embodiments, at least one of the two amino acids is a cysteine or lysine. In some embodiments, at least one of the two amino acids of a pair is a wild-type amino acid of the protein. In some embodiments, at least one of the two amino acids of a pair has been mutated from its wild-type amino acid. In some embodiments, at least one of the two amino acids of a pair is a non-natural amino acid. In some embodiments, a non-natural amino acid is mutated into the protein. In some embodiments, the non-natural amino acid is p-azido-L-phenylalanine (AZF) (e.g., replacing a native/wild-type phenylalanine). Examples of non-natural amino acids that can be used for site-specific protein labeling may include 1: 3-(6-acetylnaphthalen-2-ylamino)-2-aminopropanoic acid (Anap), 2: (S)-1-carboxy-3-(7-hydroxy-2-oxo-2H-chromen-4-yl)propan-1-aminium (CouAA), 3: 3-(5-(dimethylamino)naphthalene-1-sulfonamide) propanoic acid (Dansylalanine), 4: Nε-p-azidobenzyloxycarbonyl lysine (PABK), 5: Propargyl-L-lysine (PrK), 6: Nε-(1-methylcycloprop-2-enecarboxamido) lysine (CpK), 7: Nε-acryllysine (AcrK), 8: Nε-(cyclooct-2-yn-1-yloxy)carbonyl)L-lysine (CoK), 9: bicyclo[6.1.0]non-4-yn-9-ylmethanol lysine (BCNK), 10: trans-cyclooct-2-ene lysine (2′-TCOK), 11: trans-cyclooct-4-ene lysine (4′-TCOK), 12: dioxo-TCO lysine (DOTCOK), 13: 3-(2-cyclobutene-1-yl)propanoic acid (CbK), 14: Nε-5-norbornene-2-yloxycarbonyl-L-lysine (NBOK), 15: cyclooctyne lysine (SCOK), 16: 5-norbornen-2-ol tyrosine (NOR), 17: cyclooct-2-ynol tyrosine (COY), 18: (E)-2-(cyclooct-4-en-1-yloxyl)ethanol tyrosine (DS1/2), 19: azidohomoalanine (AHA), 20: homopropargylglycine (HPG), 21: azidonorleucine (ANL), 22: Nε-2-azideoethyloxycarbonyl-L-lysine (NEAK).

In some embodiments, at least one of the two amino acids of a pair is labeled using an N-terminal transglutaminase. In some embodiments, labeling is done between N-terminal transglutaminase and a non-natural amino acid with orthogonal chemistry (such as functional p-azido-L-phenylalanine (AZF) group).

In some embodiments, the pair or pairs of amino acids are chosen at random to replace with a non-standard amino acid (e.g. AZF). In some embodiments, all solvent-exposed native cysteines and/or lysines are labeled with FRET dyes.

In some embodiments, a researcher uses a protein structure prediction model (e.g., a coarse protein structure prediction model) to identify amino acid residues that are amenable to labeling with a FRET dye molecule. In some embodiments, a researcher uses a protein structure prediction model (e.g., a coarse protein structure prediction model) to identify amino acid residues that are amenable for mutation to introduce an amino acid (e.g., cysteine, lysine, or a non-natural amino acid) that can be labeled with a FRET dye. Amino acid residues that are amenable for labeling or mutation can be labeled or mutated without significant disruption to the conformation of the protein (e.g., are solvent-exposed, in an active site or located outside of a structural domain). In some embodiments, the protein structure prediction model is a protein folding algorithm. In some embodiments, the protein structure prediction model identifies at least one pair of amino acids on the surface of the protein for which the model cannot predict their locations (e.g., distances from one another) with a high degree of accuracy and/or precision. In some embodiments, the protein structure prediction model identifies at least one pair of amino acids that would benefit from increased resolution of their location (e.g., location of one amino acid of the pair relative to the other). In these embodiments, the protein structure prediction model first predicts the relative locations of all of the amino acids on the surface of the protein relative to one another in order to produce a distogram or distance matrix.

Once all the surface residues of the protein are identified, a single residue may be chosen for the first label. In some embodiments, this single residue is a cysteine that is not a part of a disulfide bond or a lysine. The algorithm may predict whether the single residue is an element of a stabilizing force of the protein (e.g., element of a disulfide bond). If the single residue is mutated, the algorithm will provide a listing of optional amino acids for mutation that are chemically similar to the native amino acid in order to not disrupt the conformation or stability of the protein. Then, the algorithm may draw a sphere and identify all other cysteines, lysines, or replaceable amino acids within a 10 angstrom radius. If the algorithm locates any other of these amino acids, it may again check to see whether this is a solvent-accessible amino acid. If it is, this may be chosen to be the second amino acid of the pair for labeling.

In some embodiments, in order to identify surface exposed residues, the protein structure prediction model first checks for protein loops. The protein structure prediction model may then check for possible disruption of secondary structure, and then locate all potential pairs of amino acids that can be labeled or mutated.

In some embodiments, the protein structure prediction model (e.g., protein folding algorithm) further refines the selection of a pair of amino acid by suggesting amino acid residues that maximally collapse the number of possible solution sets. In some embodiments, the algorithm determines the estimated distance between each and every possible solvent-exposed amino acid residue. In some embodiments, the algorithm then produces a distogram (or matrix of distances between each possible pair of amino acids) and rank orders each possible pairing of amino acids based on one of several factors (e.g., the uncertainty or variance in the measurement of the distance between each pairing). The algorithm may then use this ordered list of possible amino acid pairs (e.g., ranked from highest uncertainty or variance to least uncertainty or variance) to identify at least one pair of amino acids that could be labeled with a FRET dye or mutated to allow for labeling with a FRET dye. In vitro experimental determination of the distance between the two identified amino acid residues can then be used to refine the algorithm by constraining the possible distance between the pair of amino acids during subsequent predictions of the structure of the protein.

### Methods for Labeling

For a given protein, pairs of amino acids on the surface of the protein are chosen to be labeled by FRET dyes. In some embodiments, the pairs of amino acids are amenable to labeling (e.g., cysteine, lysine). In other embodiments, one or both of the amino acids of a pair is a native amino acid that is not amenable to labeling (e.g., glycine). Amino acids that are not amenable to labeling can be mutated to natural amino acids that are amenable to labeling (e.g., cysteine, lysine) or to non-natural amino acids having functional chemical groups that are amenable to labeling.

In some embodiments, amino acids are labeled with FRET dye molecules. One amino acid of a pair can be labeled with a FRET donor molecule and the second amino acid of the pair can be labeled with a FRET acceptor molecule. FRET pairs are typically chosen at an estimated distance between one and ten nanometers, and when possible (based on limited computational structure predictions) amino acid pairs should be chosen in this range for maximum accuracy. FRET dyes are typically decorated near the active site of the protein, in an inert area, or on the N or C terminus of the protein.

In some embodiments, a FRET molecule is a small organic dye, a fluorescent protein, or a quantum dot. In some embodiments, a fluorescent protein for use in FRET is as described in Bajar, B. T., “A Guide to Fluorescent Protein FRET Pairs” Sensors (Basel). 2016 September; 16(9): 1488; the entire contents of which are incorporated herein by reference. In some embodiments, a FRET pair (i.e., FRET donor and FRET acceptor) is selected from cyan fluorescent proteins (CFPs) and yellow fluorescent proteins (YFPs), green fluorescent proteins (GFPs) and red fluorescent proteins (RFPs), far-red fluorescent proteins (FFPs) and infared fluorescent proteins (IFPs), large Stokes shift fluorescent proteins (LSS FPs) and fluorescent protein acceptors, dark fluorescent proteins, and phototransformable fluorescent proteins. In some embodiments, an organic dye typically comprises aromatic groups, planar or cyclic molecules with several π bonds. Exemplary dyes include Alexa Fluor 488 (AF488), Alexa Fluor 647 (AF647), and Texas Red. Additional fluorophores utilized in some embodiments of the methods described include fluorescein, rhodamine, coumarin, cyanine, Oregon Green, other Alexa Fluor dyes besides AF488 and AF647, eosin, dansyl, prodan, anthracenes, anthtraquinones, cascade blue, Nile Red, Nile Blue, cresyl violet, acridine orange, acridine yellow, crysal violet, malachite green, BODIPY, Atto, Tracy, Sulfo Cy dyes, HiLyte Fluor, and derivatives of each thereof. Further non-limiting examples of useful dyes are known in the art (see, e.g. Stockert, J. C and Blázquez-Castro, A. Chapter 3 Dyes and Fluorochromes, Fluorescence Microscopy in Life Sciences. 2017, Bentham Science Publishers. pp. 61-95; Herman B. Absorption and emission maxima for common fluorophores, Curr. Protoc. Cell Biol. 2001, Appendix 1:Appendix 1E).

To conjugate a FRET pair onto a protein's surface, several site-specific labeling techniques may be used. These techniques may be used independently of one another or in combination. The most important factor is that only two FRET dyes are conjugated to the protein, and that the dyes are applied to surface residues so as not to disturb or unfold the protein and generate a false signal.

FRET pairs are placed on the surface of the protein using either a combination of natural and unnatural (or non-canonical) amino acids, or exclusively unnatural amino acids. Methods for decorating cysteine residues with fluorescent dyes are widely published. In some embodiments, two canonical amino acids such as cysteines or lysines, ideally on the surface of the protein, are labeled with two separate FRET dyes. For maximum control of this labeling, all native cysteines are replaced with other non-reactive amino acids such as alanine or serine so that cysteines may be introduced at specific sites in the protein. Ideally the native amino acids at these sites are similar in chemical composition to cysteine so that when they are replaced by cysteine, the protein's structure is not disturbed.

The most common way to achieve site-specific labeling is to conjugate the thiol group of cysteine and the amino group of lysine amino acid (AA) residues present in proteins with commercially available maleimide and succinimide dyes, respectively (Stephanopoulos and Francis, 2011). Labeling through cysteines is more attractive for site-specificity because of the low abundance of cysteines in most protein sequences (cysteines are the second most rare of all 20 AA). Clearly, this strategy has limitations for proteins where cysteines are critical for folding and function of the protein or where more than two native cysteines already exist in the protein chain.

Cysteines are preferred because they are less frequent in natural proteins. They are the second rarest amino acid. Lysines are still doable but less preferred because they are very frequent in natural proteins. Amine-reactive conjugates, such as succinimidyl-esters or isothiocyanates, can be used to label lysine residues or N-terminal amines. Care must be taken to not disrupt stabilizing bonds such as disulfide bonds.

An even mix of two FRET dyes is conjugated onto the two exposed cysteines for a maximum theoretical labeling efficiency of 50% (50% will have correct pairing of Donor and Acceptor dyes, i.e. AD, DA, while 25% will have AA and 25% will have DD).

In some embodiments, non-canonical amino acids are introduced to the protein. These amino acids are chosen to be bioorthogonal such that a FRET pair may be selectively conjugated onto the non-canonical amino acid, by way of a reaction such as click chemistry, but are not conjugated onto any natural amino acid. It is important the non-canonical amino acids to not overly disturb the local or global protein structure as this would defeat the purpose of precise distance measurements. Propargyllysine and p-acetylphenylalanine (AcF) are examples of unnatural amino acids. Propargyllysine is an unnatural amino acid which, when incorporated into a protein, can be exploited to attach commercially available fluorescent azide dyes through copper-catalyzed alkyne-azide cycloaddition click reaction (also known as click reaction). p-acetylphenylalanine (AcF), whose ketone functional group can be ligated with hydroxylamine dyes (Brustad et al., 2008). This reaction is optimally carried out at low pH, which makes it less attractive for some biological applications.

Single non-canonical amino acids are introduced at pairs of sites. They are encoded by recoded rarest stop codons, or by an expanded genetic alphabet. Labels are added with 50% theoretical efficiency, which is the same as cysteine labeling. Two non-canonical amino acids are introduced with orthogonal click chemistries. They are encoded by two rarest recoded stop codons, or by an expanded genetic alphabet. Labels are added with 100% theoretical efficiency and they are a combination of canonical and non-canonical amino acids.

### Measurement

Fluorescence energy transfer is understood as the transfer of energy from a donor dye to an acceptor dye during which the donor emits the smallest possible amount of measurable fluorescent energy. A fluorescent dye donor is for example excited with light of a suitable wavelength. Due to its spatial vicinity to an acceptor, this results in a non-radiative energy transfer to the acceptor. When the second dye is a fluorescent molecule, the light emitted by this molecule at a particular wavelength can be used for quantitative measurements. In some embodiments, the donor is excited and converted by absorption of a photon from a ground state into an excited state. If the excited donor molecule is close enough to a suitable acceptor molecule, the excited state can be transferred from the donor to the acceptor. This energy transfer results in a decrease in the fluorescence or luminescence of the donor and, if the acceptor is luminescent, results in an increased luminescence. The efficiency of the energy transfer depends on the distance between the donor and the acceptor molecule. The decrease in signal depends on the separation distance.

In some embodiment, FRET measurements are taken in bulk in a microtiter plate. In some embodiments, a single well in a microtiter plate contains millions of copies of the same protein and FRET-labeled amino acids. FRET measurements may be collected using an apparatus such as a plate reader to measure bulk fluorescence intensity. FRET-labeled pairs will vary from well to well.

The fluorescence intensity can be measured on any device capable of measuring fluorescence either in bulk or with single molecule resolution to determine the distance between these amino acids. Standard FRET measurement techniques are used to determine distances based on FRET intensity from either the fluorescence intensity or fluorescence lifetime. In some embodiments, a positive control (e.g., a FRET-labeled peptide having a known distance between the FRET pair) can be used to assist in defining the transfer function between FRET intensity and distance measurement.

In some embodiments, measurements are taken using FLIM (fluorescence lifetime imaging). The fluorescence lifetime of the donor fluorophore is reduced during energy transfer, a process that can be imaged using FLIM. FLIM builds an image based around differences in the exponential decay of fluorescence (i.e., fluorescence lifetime). This method is particularly useful because it can discriminate fluorescent intensity changes due to the local environment and it is insensitive to the concentration of the fluorophores.

In some embodiments, FRET measurements are taken using fluorescence anisotropy. Anisotropy measurements are based upon the rotation (rotation correlation time) of a fluorescent species within its fluorescence lifetime, described in detail. Two parameters are crucial for these measurements: the fluorescence lifetime and the size of the label. If the lifetime is too short, the population will appear highly anisotropic, whereas, if it is too long, the species will have low anisotropy. Fluorescein with a lifetime of 4 ns is useful for this application. Anisotropy measurements are particularly suited when one protein is significantly smaller than the other. When binding to the larger protein, the anisotropy of the smaller unit increases because the larger complex has a slower rotation correlation time. This provides a sensitive measurement of complex formation. However, when a large label is used, as for instance a fluorescent protein, then the rotation is inherently slow giving rise to high anisotropy values, which compromises the sensitivity of the measurements. Therefore, they should be avoided.

In some embodiments, the measurements are taken at the single molecule level in an apparatus such as a zero-mode waveguide. A zero-mode waveguide comprises discrete chambers (or wells), wherein each chamber contains a separate copy of the protein with a different FRET pair. In a zero-mode waveguide based apparatus, each protein variant with its unique label pair resides in its own chamber, and therefore, each chamber measures an independent distance measurement.

In some embodiments, the protein of interest is attached to the surface via a biotin-streptavidin link. The bottom surface of the zero mode waveguide is functionalized with a biotin tethered to a high-density PEG coating. The biotin is attached to a streptavidin intermediary, which then binds to another biotin on the surface of the protein of interest. The final attachment order is: ZMW Surface:PEG-biotin:Streptavidin:biotin-protein. A maximum of one streptavidin-bound protein must sit in each zero mode waveguide to avoid overlapping signal.

In some embodiment, the FRET pairs are measured using a conventional fluorescence microscope. In some embodiment, the FRET pairs are measured using a total internal reflection fluorescence (TIRF) microscope.

In some embodiment, FRET measurements are obtained using a dynamic structure of the protein interacting with a substrate. This would require a single molecule imaging device with time-series data collection, such as a zero mode waveguide or TIRF microscope. Once the protein variants have been bound to the imaging surface, reaction substrate can be injected at high concentration to catalyze a protein reaction or initiate a protein-substrate binding event. Because each molecule is imaged independently, the distance change in each FRET pair can be aligned via software after the measurement point. This provides a large advantage over dynamic X-ray crystallography, which requires that each protein must react with the substrate at the exact same time in order to be imaged as a single synchronized crystal. This means that a much wider variety of reaction types can be assayed beyond light-activated reversible reactions. In some embodiments, these methods enable measurement of distances involved in non-reversible reactions.

In some embodiments, the total measurement time last for 30 seconds due to inevitable photo-bleaching from the laser excitation. In some embodiments, the total measurement time lasts for 1-60, 5-60, 10-60, 20-60, or 30-60 seconds. This provides sufficient time to collect measurements to construct both the static and dynamic crystal structures. This also provides enough time to flow in a ligand of interest or otherwise change the buffer conditions to see how the protein being assayed changes conformation

### Barcoding

In some embodiments, for imaging methods where physical segregation is used to separate variants (e.g., imaging in a microtiter plate or zero-mode waveguide), the individual protein variants do not need to be barcoded (e.g., with a unique molecular identifier). In some embodiments, for imaging methods where physical segregation is used to separate variants (e.g., imaging in a microtiter plate or zero-mode waveguide), the individual protein variants are barcoded.

In some embodiments, for methods to identify which two amino acids have been labeled after the single-molecule FRET measurements have been taken, the proteins are barcoded. Barcoding of a protein variant can be done in any conceivable way known to a person of skill in the art (e.g., polypeptide sequencing).

In some embodiments, the barcode of a protein variant comprises a short, protein-bound, nucleic acid-based unique molecular identifier. In some embodiments, the barcode of a protein variant comprises a complete protein-coding nucleic acid sequence. In some embodiments, the barcode of a protein variant is its amino acid sequence.

An in vitro genotype-phenotype link can be established in several ways, including via ribosome display, direct RNA binding, mRNA display, phage display, yeast display, or via the construction of a fusion protein with a DNA-binding domain.

Depending on the type of barcode used, various readout methods may be employed. If a random nucleic acid sequence barcode is used, complementary fluorescently labeled DNA, RNA, LNA, or PNA probes can be introduced to the bulk sample at high concentration and hybridized to the unique barcodes. In order to create a great enough number of protein variants, combinations of fluorophores can be used to create unique visible signatures. This will likely limit the number of detectable protein variants to double-digits.

If a direct genotype-phenotype link is created, nucleic acid sequencing on a zero mode waveguide sensor allows for the most accurate identification of a high number of variants (thousands to millions). If ribosome display was used to link the coding RNA to the protein of interest, a reverse transcriptase reaction coupled with single-molecule DNA sequencing on a PacBio system can be employed to recover the coding DNA sequence. If a fusion DNA-binding protein is formed, direct single-molecule DNA sequencing on a PacBio system may be used to recover the DNA sequence. If no genotype-phenotype link is created, single molecule peptide sequencing may be used to identify individual amino acid residues.

### Refining the Algorithm

In some embodiments, after FRET-determined distance measurements are collected for multiple pairs of amino acids in a protein, these measurements are used to refine a distogram, wherein each entry in the matrix is a probability distribution that captures the likelihood of the distance from one amino acid to every other amino acid. In some embodiments, the most effective use of the FRET-based distance measurements is in conjunction with a computational protein folding prediction model. In some embodiments, the distogram is a component of protein folding prediction algorithms. The distogram may be combined with predicted angles between the amino acid backbone and predicted distances (e.g., with statistical uncertainty or a distogram) between each amino acid to recover a complete protein structure. The distances generated by FRET measurements, in some embodiments, act as constraints on a structure prediction algorithm (e.g., a computational protein folding model). In some embodiments, constraining the algorithm decreases the total computational time to determine the structure of a protein (e.g., by at least 10%, 20%, 30%, 40%, 50%, 75%, or 100%). In some embodiments, constraining the algorithm leads to a more accurate prediction of the structure of a protein of interest.

In some embodiments, an algorithm is a probabilistic model that generates a posterior angelogram and a distogram (e.g., a probabilistic matrix of the angles and distances, respectively, between every amino acid).

In some embodiments, the algorithm will find multiple solutions that minimize the energy landscape described by the distogram. However, once the FRET labeling provides the ground-truth distances between several locations, solution structures of a protein can be eliminated that diverge (i.e., fall outside of a specified range) from the distances measured by FRET between the amino acid residues.

In some embodiments, it is envisioned that the algorithm will be implemented by a computer processor.

### Computer Implementation

It is envisioned that at least some of the method steps described herein can be implemented by a computing processor. Software in any suitable programming language would cause a processor to implement such steps. For example, some aspects of the present disclosure provide a computer-implemented method comprising: performing in silico a three-dimensional structure prediction of a protein using a structure prediction algorithm; identifying in silico at least one pair of solvent-exposed amino acids in the protein based on algorithm-predicted factors (e.g., variance in the spatial distance between the two amino acids of the at least one pair); and constraining the structure prediction algorithm using distance measurements collected in vitro between amino acids of the at least one pair of amino acids present in a recombinant copy of the protein using fluorescence resonance energy transfer (FRET), wherein a FRET donor is attached to one amino acid of the pair and a FRET acceptor is attached to the other amino acid of the pair. It is envisioned that the software may include an artificial intelligence based machine learning algorithm, trained on data, which can learn and improve as more data is fed into the system.

Other aspects of the present disclosure provide a computer readable medium on which is stored a computer program which, when implemented by a computer processor, causes the processor to: perform in silico a three-dimensional structure prediction of a protein using a structure prediction algorithm; identify in silico at least one pair of solvent-exposed amino acids in the protein based on algorithm-predicted factors (e.g., variance in the spatial distance between the two amino acids of the at least one pair; and constrain the structure prediction algorithm using distance measurements collected in vitro between amino acids of the at least one pair of amino acids present in a recombinant copy of the protein using FRET, wherein a FRET donor is attached to one amino acid of the pair and a FRET acceptor is attached to the other amino acid of the pair.

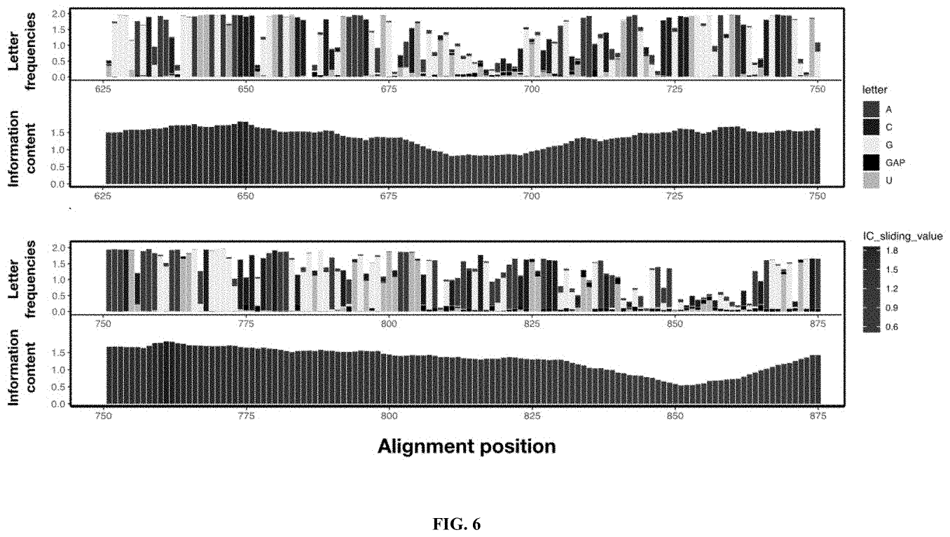

An illustrative implementation of a computer system 1400 that may be used in connection with any of the embodiments of the technology described herein is shown in FIG. 7. The computer system 1400 includes one or more processors 1410 and one or more articles of manufacture that comprise non-transitory computer-readable storage media (e.g., memory 1420 and one or more non-volatile storage media 1430). The processor 1410 may control writing data to and reading data from the memory 1420 and the non-volatile storage device 1430 in any suitable manner, as the aspects of the technology described herein are not limited in this respect. To perform any of the functionality described herein, the processor 1410 may execute one or more processor-executable instructions stored in one or more non-transitory computer-readable storage media (e.g., the memory 1420), which may serve as non-transitory computer-readable storage media storing processor-executable instructions for execution by the processor 1410.

Computing device 1400 may also include a network input/output (I/O) interface 1440 via which the computing device may communicate with other computing devices (e.g., over a network), and may also include one or more user I/O interfaces 1450, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

The above-described embodiments can be implemented in any of numerous ways. For example, the embodiments may be implemented using hardware, software or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-discussed functions of one or more embodiments. The computer-readable medium may be transportable such that the program stored thereon can be loaded onto any computing device to implement aspects of the techniques discussed herein. In addition, it should be appreciated that the reference to a computer program which, when executed, performs any of the above-discussed functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques discussed herein.

### OTHER EMBODIMENTS

Some aspects of the present disclosure provides methods comprising: (i) performing in silico a three-dimensional structure prediction of a protein using a structure prediction algorithm; (ii) identifying in silico at least one pair of solvent-exposed amino acids in the protein based on at least one algorithm-predicted factor; (iii) labeling in vitro the at least one pair of amino acids in at least one recombinant copy of the protein such that a fluorescence resonance energy transfer (FRET) donor is attached to the first amino acid of the pair and a FRET acceptor is attached to the second amino acid of the pair; (iv) collecting in vitro distance measurements between the two amino acids of the at least one pair using FRET; and (v) constraining the structure prediction algorithm using the collected distance measurements. In some embodiments, the at least one algorithm-predicted factor that allows for identification of the at least one pair of solvent-exposed amino acids is variance in the spatial distance between the two amino acids of the at least one pair, the relative importance of the distance between the two amino acids in the structure prediction algorithm and/or the structural sensitivity of the pair.

Other aspects of the present disclosure provide computer-implemented methods comprising: performing in silico a three-dimensional structure prediction of a protein using a structure prediction algorithm; identifying in silico at least one pair of solvent-exposed amino acids in the protein based on algorithm-predicted factors (e.g., variance in the spatial distance between the two amino acids of the at least one pair); and constraining the structure prediction algorithm using distance measurements collected in vitro between amino acids of the at least one pair of amino acids present in a recombinant copy of the protein using fluorescence resonance energy transfer (FRET), wherein a FRET donor is attached to one amino acid of the pair and a FRET acceptor is attached to the other amino acid of the pair.

Yet other aspects of the present disclosure provide a computer readable medium on which is stored a computer program which, when implemented by a computer processor, causes the processor to: perform in silico a three-dimensional structure prediction of a protein using a structure prediction algorithm; identify in silico at least one pair of solvent-exposed amino acids in the protein based on algorithm-predicted factors (e.g., variance in the spatial distance between the two amino acids of the at least one pair); and constrain the structure prediction algorithm using distance measurements collected in vitro between amino acids of the at least one pair of amino acids present in a recombinant copy of the protein using fluorescence resonance energy transfer (FRET), wherein a FRET donor is attached to one amino acid of the pair and a FRET acceptor is attached to the other amino acid of the pair.

In some embodiments, the methods further comprise (vi) performing in silico a three-dimensional structure prediction of a protein using the constrained structure prediction algorithm, and optionally further repeating, at least 1, 2, 3, or more times, each of (ii) to (vi).

In some embodiments, the pair of amino acids are separated based on the primary structure of the protein by at least five amino acids.

In some embodiments, (i) comprises performing in silico a three-dimensional structure prediction of a protein using a structure prediction algorithm and generating a probabilistic matrix or distogram of the distances between each combination of two amino acids in the protein.

In some embodiments, (ii) comprises determining the algorithm-predicted variance in the spatial distance between every combination of two solvent-exposed amino acids and rank-ordering every combination of two solvent-exposed amino acids based on algorithm-predicted factors, optionally wherein the at least one pair of amino acids is identified as having the largest algorithm-predicted variance in spatial distance.

In some embodiments, in (ii), the algorithm-predicted variance in the spatial distance between the two amino acids comprises a k-value of between 1 and 100.

In some embodiments, the methods comprise: (i) performing in silico a three-dimensional structure prediction of a protein using a structure prediction algorithm; (ii) identifying in silico 2, 3, 4, 5, or more pairs of solvent-exposed amino acids in the protein based on algorithm-predicted variance in the spatial distance between the two amino acids of each pair; (iii) labeling in vitro each pair of amino acids in a recombinant copy of the protein such that a fluorescence resonance energy transfer (FRET) donor is attached to the first amino acid of each pair and a FRET acceptor is attached to the second amino acid of each pair, wherein each pair of amino acids is labeled in a different recombinant copy of the protein; (iv) collecting in vitro distance measurements between the two amino acids of each pair using FRET; and (v) constraining the structure prediction algorithm using the collected distance measurements.

In some embodiments, in (iii), each different recombinant copy of the protein comprises a unique molecular identifier or barcode sequence.

In some embodiments, in (iii), each different recombinant copy of the protein is placed into an individual well of a multi-well plate or an individual chamber of a zero-mode waveguide.

In some embodiments, each different recombinant copy of the protein is attached to the bottom of an individual well of a multi-well plate or an individual chamber of a zero-mode waveguide, optionally wherein each different recombinant copy of the protein is attached via a biotin-streptavidin linkage.

In some embodiments, one of the amino acids of the at least one pair is a cysteine, a lysine, or a non-natural amino acid, optionally wherein the non-natural amino acid is p-azido-L-phenylalanine.

In some embodiments, the FRET acceptor and FRET donor are organic dyes, fluorescent proteins, or quantum dots. For example, the fluorescent proteins may be cyan fluorescent proteins (CFPs) and yellow fluorescent proteins (YFPs); green fluorescent proteins (GFPs) and red fluorescent proteins (RFPs); or far-red fluorescent proteins (FFPs) and infared fluorescent proteins (IFPs).

In some embodiments, the collecting in (iv) involves total internal reflection fluorescence, fluorescence lifetime imaging microscopy, or zero-mode waveguide sensing. In some embodiments, the collecting in (iv) is done using single-molecule methods.

In some embodiments, the at least one recombinant copy of the protein is barcoded. In some embodiments, the at least one recombinant copy of the protein is barcoded with a unique molecular identifier, optionally a nucleic acid-based or peptide-based unique molecular identifier.

Some aspects of the present disclosure provide methods of in silico mining for new homologs of a protein of interest, the method comprising producing an initial protein homolog sequence database (DBinit) for the protein of interest; generating a representative reference database (DBrep) of putative protein homolog sequences by eliminating multiple sequences in the DBinit that share at least 75% identity; screening a metagenomic read database using the DBrep as a query to identity datasets of sequencing reads, and optionally ranking the datasets to determine which are most likely to contain the highest number of true homologs; aligning the DBrep to sequencing reads of the metagenomic datasets; assembling the sequencing reads into contigs (a set of overlapping DNA segments that together represent a consensus region of DNA); translating open reading frames (ORFs) of the contigs into protein sequences having greater than a cutoff fraction of the length of the average DBrep protein sequence; aligning the translated protein sequences with the DBrep protein sequences and identifying new putative protein homolog sequences, and optionally adding the new putative protein homolog sequences to the DBinit to produce an enhanced protein homolog sequence database (DBenhanced). In some embodiments, the whole-genome metagenomic fraction of the NCBI sequencing read archive (SRA) is the metagenomic read archive that is screened using DBrep as a query.

Other aspects of the present disclosure provide computer implemented methods of mining for new homologs of a protein of interest, the method comprising: producing an initial protein homolog sequence database (DBinit) for the protein of interest; generating a representative reference database (DBrep) of putative protein homolog sequences by eliminating multiple sequences in the BDinit that share at least 75% identity; screening a whole-genome metagenomic sequencing read database using the DBrep as a query to identify datasets of sequencing reads, and optionally ranking the datasets to determine which are most likely to contain the highest number of true homologs; aligning the DBrep to sequencing reads of the whole-genome metagenomic datasets; optionally assembling sequencing reads that are shorter than a full-length sequence of the protein of interest into contigs; translating open reading frames (ORFs) of long sequencing reads and/or assembled contigs into protein sequences having greater than a cutoff fraction of the length of the average DBrep protein sequence; aligning the translated protein sequences with the DBrep protein sequences and identifying new putative protein homolog sequences, and optionally adding the new putative protein homolog sequences to the DBinit to produce an enhanced protein homolog sequence database (DBenhanced).

In some embodiments, producing a protein homolog sequence database includes searching protein family databases for proteins containing a conserved protein domain. In some embodiments, producing a protein homolog sequence database includes searching protein sequence databases using pairwise or hidden Markov model (HMM)-based alignment.

In some embodiments, the methods further comprise assessing completeness of the DBinit by aligning a known non-redundant protein reference database and the DBinit, optionally using a protein alignment tool adapted for large query sets and searching for additional homologs of the protein of interest.

In some embodiments, the DBrep is generated by clustering the DBinit at 90% using a clustering algorithm.

In some embodiments, aligning the DBrep to sequencing reads of whole-genome metagenomic datasets in a read archive comprises aligning the DBrep to a sampling of reads/read-pairs from each individual whole-genome metagenomic run, optionally wherein the sampling size is about 100,000 reads.

In some embodiments, the methods further comprise quality control steps to remove unassembled reads from the sequencing read datasets.

In some embodiments, translating comprises translating six ORFs of the contigs.

In some embodiments, the methods further comprise quality control steps to validate the putative protein homolog sequences as true protein homolog sequences, which are then optionally added to the DBenhanced.

In some embodiments, the methods further comprise target protein enrichment.

In some embodiments, the methods further comprise generating a representative multiple sequence alignment (MSA) based on the DBenhanced.

Other aspects of the present disclosure provide target enrichment methods comprising: providing a list of putative protein homolog sequences of a protein of interest from a multiple sequence alignment (MSA) of sequences homologous to the protein of interest; contacting a sample comprising DNA with probes to produce probes bound to DNA, wherein the probes are designed to hybridize, optionally with low stringency, to the nucleotide sequences of the putative protein homolog sequences, and wherein the probes are immobilized on a substrate that optionally includes a separation medium; selectively removing from the substrate probes that are not bound to DNA; sequencing the DNA bound to the probes to produce sequencing reads; aligning the sequencing reads to the MSA and assembling contigs from any sequencing reads that are shorter than the full-length sequence of the protein; translating open reading frames (ORFs) from the contigs to generate new putative protein homolog sequences, and optionally validating the new putative protein homolog sequences as true protein homolog sequences; and optionally adding the new putative protein homolog sequences to the MSA to produce an enriched MSA.

In some embodiments, the methods further comprise executing on the MSA an algorithm for deducing direct correlation, optionally wherein the algorithm is a Direct Coupling Analysis (DCA) algorithm.

In some embodiments, the methods further comprise performing feature extraction using the enriched MSA for a co-evolution-based protein structure prediction model.

Further aspects of the present disclosure provide iterative homolog discovery methods comprising: (a) performing a method of in silico mining for new homologs of a protein of interest to produce an enhanced multiple sequence alignment (MSA) as described herein; (b) performing a target enrichment method as described herein to identify new putative protein homolog sequences, wherein the DNA sample has been identified using metadata for metagenomic SRA samples with positive homolog identification; (c) adding the new putative protein homolog sequences to the enhanced MSA; and optionally repeating the steps (a)-(c) iteratively.

Some aspects of the present disclosure provide computer implemented iterative homolog discovery methods comprising: (a) performing a method of in silico mining for new homologs of a protein of interest to produce an enhanced multiple sequence alignment (MSA) as described herein; (b) processing new putative protein homolog sequences obtained by a target enrichment method as described herein, wherein the DNA sample has been identified using metadata for metagenomic SRA samples with positive homolog identification; (c) adding the new putative protein homolog sequences to the enhanced MSA; and optionally repeating the steps (a)-(c) iteratively.

Also provided herein is a computer readable medium on which is stored a computer program which, when implemented by a computer processor, causes the processor to: produce an initial protein homolog sequence database (DBinit) for the protein of interest; generate a representative reference database (DBrep) of putative protein homolog sequences by eliminating multiple sequences in the DBinit that share at least 75% identity (e.g., at least 80% or at least 90% identity); screen a whole-genome metagenomic sequencing read archive using the DBrep as a query to identity datasets of sequencing reads, and optionally rank the datasets to determine which are most likely to contain the highest number of true homologs.

In some embodiments, the computer program further causes the processor to: align the DBrep to sequencing reads of the metagenomic datasets to identify hit reads; assemble hit reads into contigs; translate open reading frames (ORFs) of the contigs into protein sequences having greater than a cutoff fraction of the length of the average DBrep protein sequence; align the translated protein sequences with the DBrep protein sequences and identifying new putative protein homolog sequences, and optionally add the new putative protein homolog sequences to the DBinit to produce an enhanced protein homolog sequence database (DB enhanced).

Additional aspects of the present disclosure provide a computer readable medium on which is stored a computer program which, when implemented by a computer processor, causes the processor to: align sequencing reads to a multiple sequence alignment (MSA) and assembling contigs from any sequencing reads that are shorter than a full-length sequence of the protein; translating open reading frames (ORFs) from the contigs to generate new putative protein homolog sequences; and add the new putative protein homolog sequences to the MSA to produce an enriched MSA.

### Example 1. In Silico Mining for New Protein Homologs

The sequencing read archive (SRA) is a partially publicly accessible archive of most of the world's Next-Gen Sequencing (NGS) data, carrying a massive amount of genetic information, including the sequences of naturally-occurring proteins homologous to a protein of interest. Specifically, the set of >110,000 “whole-genome metagenomic” NGS datasets (“runs”) holds the (partial) sequences of >1.5×10¹² randomly-sampled DNA fragments from communities of microbes isolated across the globe from various ecosystems and host organisms (these sequencing “reads” are typically 100-250 bases in length, often coming in pairs constructed from the 2 ends of a fragment, but in rarer cases can extend to several kilobases).

The methods herein apply SRA mining for the purposes of assembling a superior MSA for protein structure prediction. No protein structure prediction software to date uses an MSA building approach that is compatible with raw nucleic acid sequencing read datasets such as those in the SRA. The bigger and more diverse an MSA is, the higher the quality of the DCA that can be performed, the more precise the generated contact map estimation, and the more accurate the 3D structure prediction.

SRA mining was performed to discover as many homologs of the Phi29 DNA polymerase as possible using the following protocol. The results are captured in FIGS. 3A and 3B.

An initial database (DBinit) was composed of 29 unique DNA polymerase sequences known to be homologs of Phi29 DNA polymerase. The completeness of DBinit was assessed by downloading the entire NCBI non-redundant (nr) protein reference database and using it as a query against the DBinit initial database using DIAMOND, a fast and sensitive protein alignment tool adapted for large query sets, to search it for additional hits. There were 12,326 unique query hits against DBinit in the NCBI non-redundant database (default parameters). To eliminate false positive hits, (i) the score of the hit against DBinit and (ii) the maximum possible score (e.g., self-hit) were calculated for each of the 12,326 unique polymerase query hits. Of the 12,326 query hits, 25 Phi29-like sequences were determined to be “real” hits by the Blast Score Ratio. All 25 full-length phi29 DNA polymerase homolog protein sequences were appended to the DBinit, increasing its size to a total of 54 unique sequences.

The 54 phi29-like DNA polymerase sequences in DBinit were then clustered at 90% identity using UCLUST to generate a reference database (DBrep) consisting of 30 representative Phi29-like DNA polymerase protein sequences. Searchsra with DBrep was then run as the database using the public searchsra.org service to sample 100,000 reads/read-pairs from each of the ˜107,000 “whole-genome metagenomic” runs in the SRA processed by searchsra.org (as of October 2019), revealing 369,913 read hits over 25,440 individual SRA runs (datasets). 10 of the SRA run datasets that returned the most read hits from the 100,000-read sampling were manually downloaded, formatted and cleaned. Of these 10 datasets, the 7 datasets containing paired-end reads (better for contig assembly) were selected for further analysis. For each of the 7 SRA run datasets, all reads were searched against the DBrep database and the same ultra-fast DNA-protein aligner as searchsra.org: DIAMOND. For each dataset, full-length hit reads were assembled de novo into contigs using an Iterative de Bruijn Graph Assembler optimized for metagenomic data (IDBA-UD).

Open Reading Frames (ORFS) resulting in protein sequences >70% the length of the average Phi 29 pol DB member were then translated from these contigs in all 6 reading frames. The translated ORFs in all 6 frames were aligned directly to DBrep to find protein sequences (putative new homologs) aligning over 70% of the length of a DBrep member sequence. A final stringency step (see Step 12 above) was then performed to ensure that detected homologs were closer to a member of the complete DB (DBinit) than to any other of the world's known proteins, revealing 13 brand-new, diverse phi29 DNA polymerase protein homologs. New homologs were added to DBinit, generating an enhanced homolog listing, or DBenhanced.

### Example 2. Target Enrichment for New Protein Homologs

Target enrichment sequencing involves the pre-treatment of a DNA to enrich for sequences that resemble a given target such that upon sequencing, fewer sequencing reads are required to fully enumerate all variants in the complex mixture with high coverage, which would otherwise be most costly and time-consuming for a non-enriched sample.

To “mine” physical DNA samples for nucleic acid sequences that code for proteins homologous to a target of interest, one can perform steps listed. The methods provided herein use target enrichment for the purposes of assembling a superior MSA for protein structure prediction. No protein structure prediction software uses physical, experimental methodology for constructing an MSA. The bigger and more diverse an MSA is, the higher quality DCA that can be performed, the more precise the generated contact map estimation, and the more accurate the 3D structure prediction.

There are multiple target enrichment strategies, but one in particular, called Scodaphoresis, is particularly attractive for mining homologs from physical samples. Provided herein is modified scodaphoresis for target enrichment of divergent homologs, where the design of probe sequences and target enrichment conditions is intentionally manipulated to enrich as many sequence variants as possible with relaxed stringency.

Below is a description of the methods used to enrich Phi29-like genes from a soil sample by scodaphoresis, as well as figures describing the data and analyzed results.
1. Environmental DNA was extracted from wet soil at 351A New Whitfield St, Guilford, Conn. 06437 using the PowerSoil DNeasy Pro kit. The manufacturer's instructions were followed.
2. Soil DNA was simultaneously fragmented down to 1-3 kb and appended with adapters using the tagmentation method.
3. 8 known Phi29 homologs (2 kb in length) that range in Phi29 homology from 40-100% were spiked into the tagmented soil DNA sample at low abundance (1:1000 mass ratio) >these serve as positive controls for enrichment and enable quantification of enrichment as a function of % homology.
4. Spiked soil sample was enriched for Phi29 using two different scodaphoresis methodologies (see FIG. 11), while a control sample was not enriched.
5. Scodaphoresis consisted of the following general steps:
   a. Capture tagmented, spiked soil sample in separation medium containing immobilized Phi29 probe set. “Off target” (highly mobile) sequences will flow through the separation medium and be removed at this stage.
   b. Release previously low mobility, gel-immobilized, enriched sequences by a step change elevation in the temperature.
      i. Recovery of enriched sequences that are highly mobile is possible at elevated temperature by their electrophoresis out of the gel-like matrix.
      ii. Enriched sequences can be recovered from an extraction port.
      iii. Program a series of gradual step changes in temperature to selectively release one or more enriched nucleic acid sequences according to their hybridization binding energy to the immobilized phase.
      iv. With perpendicular electric fields, switch directions of the electrophoresis driving force to run enrichment in series where the low-mobility material that remains in the gel after one round of enrichment is the starting material for a subsequent round.
      v. Use of dynamic, rotating electric fields to drive synchronous coefficient of drag alteration (SCODA) electrophoresis to finely differentiate nucleic acid variants according to slight differences in their mobilization at different temperatures.
6. Library prep (SMRTBell Template Prep kit 1.0) and long-read, circular consensus PacBio sequencing.
7. Long read, circular consensus sequencing and analysis on enriched v. unenriched samples.

Across all samples, insert sizes were 1-3 kb (as expected from tagmentation results) and median read lengths approached 30 kb. That means that circular consensus was performed on 10-20 passes for very high accuracy reads (FIG. 13)

Interestingly, the insert size distribution changed after enrichment such that a strong peak at 2 kb emerged, as marked by arrows in FIG. 13. This reflects that the 2 kb positive control homologs that were spiked into the soil sample were so strongly enriched that they represent a large fraction of the inserts and show up prominently at a single length in the insert length distribution.

Next, it was determined what kinds of protein-coding sequences were in the unenriched soil DNA sample and how the distribution of those proteins changed after enrichment. For each 1-3 kb circular consensus sequence, all 6 frames were translated and identified the presence of conserved protein domains in the resulting open reading frames. Prior to enrichment, the most abundant protein domains are related to signaling and transport across the membrane among other putative functions. DNA polymerases of the family B type represented just 0.03% of the protein domains in the unenriched sample and were only present in the unenriched due to positive control Phi29 homologs spike-in—no Phi29 homologs outside of spiked-in controls were identified in the unenriched sample.

After enrichment, family B DNA polymerases represent 44% of the protein domains identified among the OnTarget and DeepMining enriched samples, reflecting a strong level of enrichment at the protein domain level (˜1000×).

By spiking in 8 different known Phi29 homologs of varying % homology to Phi29 at low abundance in the unenriched sample, fold changes for individual homologs were quantified and functional differences between the OnTarget and DeepMining strategies were determined.

Importantly, all 8 homologs were detected in both enrichment samples. It was found that enrichment of the homologs varied—from as low as 4 fold enrichment of AP50 (42% homology to Phi29) by DeepMining to >1400 fold enrichment of B103 (75% homology to Phi29) by OnTarget enrichment.

When the enrichment performance of OnTarget and DeepMining were compared head-to-head, an interesting trend was observed (FIGS. 15A-15B). OnTarget excelled at enriching sequences with high (75-100%) homology to Phi29 (5-10 fold better than DeepMining), and it also, surprisingly outperformed DeepMining for the lowest homology sequences. DeepMining was slightly superior to OnTarget (1.5-5 fold better) at enriching 3 of the 4 medium homology sequences.

Because the intention of enrichment is for new homolog discovery, it was desirable to look for the presence of Phi29 homologs beyond those that were intentionally added as spike-in controls.

One new Phi29 homolog—OT102800 (FIG. 16)—was identified among the OnTarget enriched sequences and added to the Phi29 gene family phylogenetic tree (FIG. 16). Finding one new homolog from 1 μg of starting soil DNA validated this approach.

As described by FIGS. 14 and 16, the new homolog is 40% homologous to Phi29 at the nucleotide level and once translated, the environmental fragment aligns to Phi29 from the Palm region through the end of the polymerase. Although the homolog was identified from a single sequencing read, accuracy for the molecule was high (57 ccs passes).

Next steps include designing primers to amplify OT102800 directly from the original soil sample by PCR to confirm its presence and determine the full length sequence.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between the upper and lower ends of the range are specifically contemplated and described herein.

What is claimed is:

1. A method comprising:
(i) generating a multiple sequence alignment (MSA) of sequences homologous to a protein of interest;
(ii) performing in silico a three-dimensional structure prediction of the MSA using a structure prediction algorithm to produce a distogram;
(iii) identifying in silico at least one pair of solvent-exposed amino acids in the MSA;
(iv) determining in vitro the distance between the two amino acids of the at least one pair; and
(v) constraining the structure prediction algorithm using the collected distance measurements.

2. The method of claim 1, further comprising:
(vi) performing in silico a three-dimensional structure prediction of a protein using the constrained structure prediction algorithm, and optionally further repeating, at least 1, 2, 3, or more times, each of (ii) to (vi).

3. The method of claim 1, wherein determining in vitro the distance between the two amino acids of the at least one pair is performed using fluorescence resonance energy transfer (FRET) experiments.

4. A system for predicting protein structures based on protein sequences, the system comprising:
at least one hardware processor;
at least one non-transitory computer-readable storage medium storing processor-executable instructions that, when executed by the at least one hardware processor, cause the at least one hardware processor to perform:
generating a contact map using a direct coupling analysis algorithm based on homologs of an input protein sequence;
providing the generated contact map to a first machine learning model as input, to generate a resulting distogram;
determining, based at least on the resulting distogram, at least one pair of solvent-exposed amino acids in the protein for which the distance between the two amino acids is to be measured using an in vitro experiment;
importing in vitro measured distances;
generating an updated distogram, that is constrained by the in vitro measured distances; and
based on the updated distogram, generating a predicted protein structure.

5. The system of claim 4, wherein generating the predicted protein structure is performed with a second machine learning model.

6. The system of claim 4, wherein the in vitro experiment is a fluorescence resonance energy transfer (FRET) experiment.

7. A method for performing directed evolution of proteins, using the system of claim 4, the method comprising iteratively performing:
producing a library of protein sequences based on an input protein structure, using a generative machine learning model configured to generate protein sequences having protein structures similar to an input protein structure;
expressing the protein sequences of the library of protein sequences;
selecting and amplifying at least a portion of the expressed protein sequences;
providing the selected and amplified protein sequences as input to the system, to output a predicted protein structure.

8. A method of training a machine learning model for distogram prediction, the method comprising:
using at least one computer hardware processor to perform:
accessing a plurality of target distograms, wherein each target distogram of the plurality of target distograms represents a target output of the machine learning model;
accessing a plurality of input contact maps, wherein each input contact map of the plurality of input contact maps:
corresponds to a target distogram of the plurality of target distograms and represents an input to the machine learning model for the corresponding target distogram; and
comprises an output of a direct coupling analysis algorithm, based on homologs of an input protein sequence; and
training the machine learning model using the plurality of target distograms and the plurality of input contact maps corresponding to the plurality of target distograms, to obtain a trained machine learning model.

9. The method of claim 8, wherein training the machine learning model comprises:
providing a first input contact map of the plurality of contact maps to the machine learning model;
providing a first target distogram of the plurality of target distograms to the machine learning model, wherein the first target distogram corresponds to the first input contact map;
obtaining, from the machine learning model, a first predicted distogram based on the first input contact map;
determining, based on the first predicted distogram and the first target distogram, a first error; and
based on the first error, updating the machine learning model.

10. A computer readable medium on which is stored software which when implemented by a processor causes that processor to perform the steps of:
(i) generating a multiple sequence alignment (MSA) of sequences homologous to a protein of interest;
(ii) performing in silico a three-dimensional structure prediction of the MSA using a structure prediction algorithm to produce a distogram;
(iii) identifying in silico at least one pair of solvent-exposed amino acids in the MSA;
(iv) determining in vitro the distance between the two amino acids of the at least one pair; and
(v) constraining the structure prediction algorithm using the collected distance measurements.

11. A computer system configured to perform:
accessing a plurality of target distograms, wherein each target distogram of the plurality of target distograms represents a target output of the machine learning model;
accessing a plurality of input contact maps, wherein each input contact map of the plurality of input contact maps:
corresponds to a target distogram of the plurality of target distograms and represents an input to the machine learning model for the corresponding target distogram; and
comprises an output of a direct coupling analysis algorithm, based on homologs of an input protein sequence; and
training the machine learning model using the plurality of target distograms and the plurality of input contact maps corresponding to the plurality of target distograms, to obtain a trained machine learning model.

12. The system of claim 11, wherein training the machine learning model comprises:
providing a first input contact map of the plurality of contact maps to the machine learning model;
providing a first target distogram of the plurality of target distograms to the machine learning model, wherein the first target distogram corresponds to the first input contact map;
obtaining, from the machine learning model, a first predicted distogram based on the first input contact map;
determining, based on the first predicted distogram and the first target distogram, a first error; and
based on the first error, updating the machine learning model.
